(19) United States
(12) Patent Application Publication
(10) Pub. No.: US 2013/0237556 A1
(43) Pub. Date: Sep. 12, 2013

(54) BERBERINE ALKALOID AS A MEDICAMENT FOR PREVENTION AND TREATMENT OF NEURAL DISEASE

(71) Applicant: HONG KONG BAPTIST UNIVERSITY, Hong Kong (HK)

(72) Inventors: Min LI, Hong Kong (HK); Siva Sundara Kumar DURAIRAJAN, Hong Kong (HK); Liangfeng LIU, Hong Kong (HK); Jiahong LU, Hong Kong (HK)

(73) Assignee: HONG KONG BAPTIST UNIVERSITY, Hong Kong (HK)

(21) Appl. No.: 13/798,066

(22) Filed: Mar. 12, 2013

Related U.S. Application Data
(60) Provisional application No. 61/609,917, filed on Mar. 12, 2012.

(51) Int. Cl. A61K 31/4745

(52) U.S. Cl. CPC A61K 31/4745
USPC 514/280; 546/48

(57) ABSTRACT

The present invention is directed to method of preventing or treating neural disease or inhibiting accumulation of β-amyloid plaques in a subject by administering an effective dosage to the subject an agent (berberine) which inhibits the accumulation of β-amyloid- plaque, phospho-amyloid precursor protein (p-APP) and tau hyperphosphorylation in a mammalian brain.

### CROSS-REFERENCE TO RELATED APPLICATIONS

The present application claims priority of U.S. provisional application No. 61/609,917 filed Mar. 12, 2012, and which the disclosure is hereby incorporated by reference.

### FIELD OF INVENTION

The present invention relates to a berberine (5,6-dihydro-9,10-dimethoxybenzo[g]-1,3-benzodioxolo[5,6-a]quinolizinium) alkaloid and the use thereof as therapeutic agent, particularly in the prevention and treatment of neural disease. In particular, the invention is directed to therapeutic effect of berberine on Alzheimer's disease by reducing Aβ deposits, tau hyperphosphorylation, gliosis and cognitive impairments.

### BACKGROUND OF THE INVENTION

Cerebral deposition of amyloid plaques containing amyloid β-peptide (Aβ) is now considered as the central feature of Alzheimer's disease (AD). Aβ, consisting of 39-43 amino acids, is a proteolytic product of a much larger amyloid precursor protein (APP). APP is an integral membrane protein cleaved either by α-secretase leading to a non-amyloidogenic pathway, or by β-secretase (BACE-1), and subsequently by γ-secretase leading to an amyloidogenic pathway. When the latter occurs, the Aβ generated by the amyloidogenic pathway triggers a series of events leading to the development of AD. In the non-amyloidogenic pathway, α-secretase cleaves APP within Aβ domain thereby not only precluding the formation of Aβ but also generating a soluble, neurotrophic and neuroprotective N-terminal ectodomian, termed sAPPα, and the 83-residue carboxyl-terminal fragment (CTF) CTF-α or CTF-C83. The action of amyloidogenic BACE-1 on APP results in the amino-terminal extracellular product sAPP-β and the carboxyl-terminal product CTFβ or C99-CTF retained in the membrane. CTFβ and CTFα are subsequently cleaved by γ-secretase to produce either Aβ (40/42) or P3, respectively, and a cytoplasmic tail dubbed APP-intracellular domain (AICD). Thus, APP proteolysis is the fundamental process for the production of Aβ peptides implicated in AD pathology. Several studies, such as da Cruz e Silva, E. F., da Cruz e Silva O. A. B., 2003. Protein phosphorylation and APP metabolism. Neurochem. Res. 28, 1553-1561 and Lee, M. S., Kao, S. C., Lemere, C. A., Xia, W., Tseng, H. C., Zhou, Y., Neve, R., Ahlijanian, M. K., Tsai, L. H., 2003a. APP processing is regulated by cytoplasmic phosphorylation. J. Cell Biol. 163, 83-95 have revealed that APP maturation and targeting for proteolysis requires APP Thr668 phosphorylation. Phosphorylated APP (p-APP) undergoes fast anterograde axonal transport to the nerve terminals, where β- and γ-secretase mediated-cleavage occurs, resulting in Aβ. Therefore, screening of agents with modulating effect on APP processing to reduce Aβ secretion levels is a promising approach to finding a new agent with which to treat, or prevent, AD.

Transgenic mice that overexpress human APP (hAPP) are playing a key role in ongoing attempts to understand the pathology of, and to develop therapeutics for, AD. The TgCRND8 mouse is one of the most well characterized strains of hAPP transgenic mice, which express hAPP695 with the Swedish and Indiana mutations. When TgCRND8 mice are three months of age, Aβ deposits become visible in their cortical brain and hippocampal regions together with astrocytic activation, microglial activation, neuritic dystrophy, inflammation and behavioral deficits that closely resembles human AD.

Treatment strategies for AD based on the amyloid hypothesis mainly involve β- and/or γ-secretase inhibitors and anti-Aβ vaccination; however, there are still unresolved issues with clinical application. Therefore, there is a need for alternative drugs. Based on advances reported in Quinn, J., Kaye, J., Montine, T., Stackman, R., 2004. Phytochemicals in Alzheimer's disease: The Development of Clinical Trials. Pharmaceu. Biol. 42, 64-73, which reported on the treatment of AD using herbs, phytotherapy seems to hold promise. One potential phytotherapeutic agent for AD could be berberine (BBR) because BBR has shown its safety and efficiency in human and animals. Berberine (5,6-dihydro-9,10-dimethoxybenzo[g]-1,3-benzodioxolo[5,6-a]quinolizinium) is an isoquinoline alkaloid that has been isolated from Coptis chinensis Franch. (Huanglian, Chinese goldthread) found in China. Coptis chinensis has been used in Chinese medicine with a long history of clinical benefits. BBR is one of the important components of Oren-gedoku-to (Huang Lian Jie Du Tang) extract, which has been used in clinical therapies for several types of dementia in China and Japan. Studies such as Kong, W., Wei, J., Abidi, P., Lin, M., Inaba, S., Li, C., Wang, Y., Wang, Z., Si. S., Pan. H., Wang, S., Wu, J., Wang, Y., Li, Z., Liu, J., Jiang, J. D., 2004. Berberine is a novel cholesterol-lowering drug working through a unique mechanism distinct from statins. Nat. Med. 10, 1344-1351, have demonstrated that BBR is an anti-inflammatory, anti-diabetic, anti-cholesterol, anti-obesity, anti-ischemic and cardioprotective compound in publications such as Lau C W, Yao X Q, Chen Z Y, Ko W H, Huang Y (2001). Cardiovascular action of berberine. Cardiovasc Drug Rev 19: 234-244, Lee Y S, Kim W S, Kim K H, Yoon M J, Cho H J, Shen Y (2006). Berberine, a natural plant product, activates AMP-activated protein kinase with beneficial metabolic effects in diabetic and insulin-resistant states. Diabetes 55: 2256-2264, Jeong, H. W., Hsu, K. C., Lee, J. W., Ham, M., Huh, J. Y., Shin, H. J., Kim, W. S., Kim. J. B., 2009. Berberine suppresses proinflammatory responses through AMPK activation in macrophages. Am. J. Physiol. Endocrinol. Metab. 296, 955-964, and Kim W S, Lee Y S, Cha S H, Jeong H W, Choe S S, Lee M R et al (2009) Berberine improves lipid dysregulation in obesity by controlling central and peripheral AMPK activity. Am J Physiol Endocrinol Metab 296: 812-819. Recent reviews such as Ye, M., Fu, S., Pi, R., He, F., 2009. Neuropharmacological and pharmacokinetic properties of berberine: a review of recent research. J. Pharm. Pharmacol. 61, 831-837, Kulkarni, S. K, Dhir, A., 2010. Berberine: a plant alkaloid with therapeutic potential for central nervous system disorders. Phytother. Res. 24: 317-324 and Ji, H. F., Shen, L., 2011. Berberine: a potential multipotent natural product to combat Alzheimer's disease. Molecules 16, 6732-6740, also have indicated that BBR has a well-documented neuroprotetcive effect against cerebral ischemia, mental depression, schizophrenia, anxiety and AD. Recently, the anti-AD effects of BBR have been mainly demonstrated in Zhu, F., Qian, C., 2006. Berberine chloride can ameliorate the spatial memory impairment and increase the expression of interleukin-1beta and inducible nitric oxide synthase in the Aβ infused rat model of Alzheimer's disease. BMC Neurosci. 7, 78., Yu, C. J., Zheng, M. F., Kuang, C. X., Huang, W. D., Yang, Q., 2010. Oren-gedoku-to and its constituents with therapeutic potential in Alzheimer's disease inhibit indoleamine 2, 3-dioxygenase activity in vitro. J. Alzheimers Dis. 22, 257-266, Jung, H. A., Min, B. S., Yokozawa, T., Lee, J. H., Kim, Y S., Choi, J. S., 2009. Anti-Alzheimer and antioxidant activities of Coptidis Rhizoma alkaloids. Biol. Pharm. Bull. 32, 1433-1438 and Shi, A., Huang, L., Lu, C., He, F., Li, X., 2011. Synthesis, biological evaluation and molecular modeling of novel triazole-containing berberine derivatives as acetylcholinesterase and β-amyloid aggregation inhibitors. Bioorg. Med. Chem. 19, 2298-2305, through acetyl and butyl cholinesterase inhibition, indoleamine 2,3-dioxygenase inhibition, amelioration of Aβ1-40-induced cognitive impairments and inhibition of Aβ1-42 fibril formation. These findings suggested that BBR is a multifunctional compound with neuroprotective properties. It has also been reported in Asai, M., Iwata, N., Yoshikawa, A., Aizaki, Y, Ishiura, S., Saido, T. C., Maruyama K, 2007. Berberine alters the processing of Alzheimer's amyloid precursor protein to decrease Abeta secretion. Biochem. Biophys. Res. Commun. 352, 498-502 that BBR reduces amyloid plaque production in Swedish APP-expressing cells; however, the in vivo efficacy of BBR on Aβ clearance is not yet validated. Thus, the effective dosage of BBR for prevention and treatment of Aβ deposits induced, tau hyperphosphorylation, gliosis, cognitive impairments and related neural diseases is still unknown.

US application no. US 2004/0097534 A1 disclosed one use of BBR against apoptosis of neuronal stem cells and differentiated neuronal stem cells, an effect of inducing the regeneration of nerve cells, a regenerative effect on neurites, a neuroregenerative effect on central nerves and peripheral nerves, a reformation effect on neuromuscular junctions, and a protective effect against apoptosis of nerve cells and a neuroregenerative effect in animals suffering from dementia and brain ischemia, but the invention disclosed in this application is novel and not known in the art.

In this study, the present application discloses, for the first time, an effective dosage of chronic administration of BBR that reduces Aβ deposits, tau hyperphosphorylation, gliosis and cognitive impairments in TgCRND8 mice.

Citation or identification of any reference in this section or any other section of this application shall not be construed as an admission that such reference is available as prior art for the present application.

### SUMMARY OF INVENTION

The first embodiment of the present invention relates to a composition comprising a compound of formula I

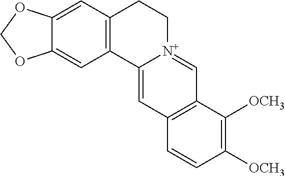 [SMILES: COc1ccc2cc3[n+](cc2c1OC)CCc1cc2c(cc1-3)OCO2]

which is berberine (BBR) for treatment of neural diseases associated with abnormal proteins aggregation or deposit in the nervous system, especially among the neuronal cells. This compound is a kind of isoquinoline alkaloid capable of being isolated from the Coptis chinensis and Berberis species.

In one embodiment there is provided a composition for the prevention, treatment or amelioration of symptoms of Alzheimer's disease (AD) comprising a berberine alkaloid, the salts and/or derivatives thereof, or a combination thereof. Examples of salts of berberine and related compounds, includes but are not limited to berberine chloride, berberine hydrochloride, berberine sulfates, berberrubine, palmatine chloride, palmitine, oxyberberine, epiberberine, dihydroberberine, tetrahydroberberine, jatrorrhizine, coreximine. The berberine alkaloid may be derived from salts, derivatives of berberine or related compounds.

In yet another embodiment of the present invention, the present invention is directed to a method of preventing or treating Alzheimer's disease in a subject by administering to the subject an composition comprising BBR that inhibits the accumulation of amyloid-β plaque and tau phosphorylation in a mammalian brain. Another embodiment of the present invention relates to elucidating the mechanism of BBR in N2a cell expressing Swedish mutant APP. The main novelties of the present invention are: (1) BBR of the present invention readily passes through the blood-brain barrier in order to target the cells/tissue with the problem of abnormal proteins aggregation or deposit. The presence of BBR in brain tissue of treated mice was definitively determined by liquid chromatography-mass spectrometry. (2) Dose-specific effects of the present invention on brain Aβ levels in TgCRND8 mouse model of Alzheimer's disease with cognitive deficits, amyloid neuropathology and accelerated gloisis. (3) The present invention significantly reduces Aβ and CTFs, by down-regulating the phosphorylation of APP and of CTFs via the activation of phosphatidylinositol 3-kinases (PI3K)/protein kinase (Akt)/Glycogen synthase kinase 3 (GSK3) pathway. (4) The present invention reduces the “hotspot” phosphotau epitopes recognized by PHF-1, AT8 and AT180 in the brain homogenates of TgCRND8 mice treated with the present invention. (5) The present invention significantly reduces the level of p-APP, CTFs and PHF-1-tau from the N2a mouse neuroblastoma cell expressing Swedish mutant APP via the activation of PI3K/Akt/GSK3 pathway. (6) The inventors of the subject application are the first to show, in immunocytochemical analysis, BBR markedly reduces p-APP staining in endosomal compartments. (7) BBR-induced reduction of p-APP is significantly inhibited by LY294002 (PI3K inhibitor) in endosomal compartments of N2a-SwedAPP cells.

Those skilled in the art will appreciate that the invention described herein is susceptible to variations and modifications other than those specifically described.

The invention includes all such variation and modifications. The invention also includes all of the steps and features referred to or indicated in the specification, individually or collectively, and any and all combinations or any two or more of the steps or features.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of and “consists essentially of' have the meaning ascribed to them in U.S. Patent law, e.g., they allow for elements not explicitly recited, but exclude elements that are found in the prior art or that affect a basic or novel characteristic of the invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

Other definitions for selected terms used herein may be found within the detailed description of the invention and apply throughout. Unless otherwise defined, all other technical terms used herein have the same meaning as commonly understood to one of ordinary skill in the art to which the invention belongs.

Other aspects and advantages of the invention will be apparent to those skilled in the art from a review of the ensuing description.

### BRIEF DESCRIPTION OF THE DRAWINGS

The above and other objects and features of the present invention will become apparent from the following description of the invention, when taken in conjunction with the accompanying drawings, in which:

FIG. 1 shows chronic BBR treatment is highly tolerable and does not influence body weight of the treated subject.

FIG. 2 show that BBR ameliorates cognitive deficits in TgCRND8 mice. BBR treatment does not significantly affect motility or vision in the treated mice in visible platform (FIG. 2A). The treated group shows significantly reduced swim path when compared with the control group in hidden platform (FIG. 2B). In the 3 interpolated probe trials, the BBR-treated TgCRND8 mice exhibit the highest duration than the vehicle-treated TgCRND8 in the target quadrant in the probe trial 2 and 3 (FIG. 2C). The asterisks denote differences between the given groups over all testing days, *p<0.05; **p<0.01; ***p<0.001 (ANOVA for repeated measures).

FIG. 3 show BBR reduces cortio-hippocampal Aβ plaque pathology in TgCRND8 mice. β-amyloid immunostained with an anti-β-amyloid(4G8) (FIG. 3A). BBR treatment at concentration of 25 mg/Kg/d results in a 60% decrease in plaque burden compared with control animals, whereas 100 mg/Kg/d of BBR treatment reduced Aβ plaque burden to 33% of untreated control (FIG. 3B). FIG. 3C shows BBR (25 mg/Kg)-treatment in TgCRND8 mice significantly reduce in large (>50), medium (25-50 μm), small sized (<25 μm) and total amyloid plaque subsets. The percentage reduction in large, medium, small and total subsets are 60%, 41%, 35% and 37%, respectively. BBR (100 mg/Kg/d) dose produces only a non-significant 21-28% reduction of amyloid plaque subsets. Values represent group mean±SEM; n=6-7 mice per group. In 4b, ***p<0.001; **p<0.01; *p<0.05 versus control after one-way ANOVA followed by Fisher LSD post-hoc analysis. In 2c, ***p<0.001; *p<0.05 versus control after two-way ANOVA followed by Fisher LSD post-hoc analysis.

FIG. 4 show BBR reduces parenchymal and cerebrovascular Aβ plaque pathology in the TgCRND8 mice. Thioflavin S-positive fibrillar parenchymal Aβ (FIG. 4Ai) and cerebral fibrillar amyloid blood vessel (FIG. 4Aii) are significantly different between treatment groups. Arrows indicates vascular amyloid and parenchymal amyloid. Digital images from cortex and hippocampus were obtained and analyzed with Image J software (NIH Image). Scale bar, 100 μm. Percentage of Aβ-positive parenchymal (FIG. 4B) and Aβ-positive perivascular vessel walls (FIG. 4C) related to the total area of interest. ***p<0.001; **p<0.01; *p<0.05 versus control after one-way ANOVA followed by Fisher LSD post-hoc analysis

FIG. 5 show BBR treatment alters Aβ peptide levels in TgCRND8 mice. TgCRND8 mice are orally administered with BBR (25 or 100 mg/Kg/d) or vehicle for 4 months. SDS-soluble and formic acid-extractable Aβ40 and Aβ42 levels from cerebral hemispheres are measured by sandwich ELISA. SDS-solubilized Aβ40 and Aβ42 levels are illustrated in FIG. 5A. FIG. 5B is Formic acid-extractable Aβ40 and Aβ42 levels. In the SDS-soluble fraction, the diminution of Aβ1-40 is 44% (p<0.05) and 32% (p=0.051) by 25 mg/Kg/d and 100 mg/Kg/d, respectively, and there is slight reduction of Aβ1-42 by BBR treatments. In the formic acid extractable fraction, the reductions of Aβ1-40 are 49% (p<0.01) and 54% (p<0.01), and the corresponding reductions of Aβ1-42 are 44% (p<0.01) and 39% (p<0.05) by 25 mg/Kg/d and 100 mg/Kg/d of BBR, respectively. Values denotes group mean±SEM. N=6-7 mice per group. *p<0.05, **p<0.01 versus vehicle control group.

FIG. 6 show BBR reduces microgliosis (FIG. 6Ai) and astrogliosis (FIG. 6Aii) in the TgCRND8 mice. BBR significantly reduces the Iba-1 burden to 45% and 27% in 25 and 100 mg/Kg/d of BBR-treated TgCRND8 mice relative to vehicle-treated TgCRDN8 mice (FIG. 6B). The Iba-1 burden (%) (FIG. 6B) and the GFAP burden (%) (FIG. 6C) are calculated by quantitative image analysis. Scale bar: 100 μm. Values denotes group mean±SEM. N=6-7 mice per group. *p<0.05 and ***p<0.001 versus vehicle control group. BBR significantly reduces the GFAP burden to 54% and 28% in 25 and 100 mg/Kg of BBR-treated TgCRND8 mice, respectively, relative to vehicle-treated TgCRND8 mice (FIG. 6C).

FIG. 7 show alteration of APP-CTFs, phosphorylation of APP, p-CTFs and tau, and expression of GSK3β in TgCRND8 mice by BBR treatment. FIG. 7A are immunoblots demonstrating the levels of full-length (Fl) APP, CTFs (CTF-α and CTFβ), p-APP(Thr668) and p-CTFs. FIG. 7B is a densitometric analysis of the ratio of Fl-APP, CTFs, p-APP and p-CTFs in the brain homogenates of TgCRND8 mice treated with BBR (25 and 100 mg/Kg/d) or vehicle. β-actin is used as a loading control. FIG. 7C are representative immunoblots of phosphorylated-tau at PHF-1, AT8 and AT180, dephosphoryaled Tau-1 and total tau Tau-5. FIG. 7D is a densitometric analysis of the ratio of PHF-1, AT8, AT180 and Tau-1. FIG. 7E is a representative immunoblots of phosphorylated GSK3 (Ser21/9), GSK3α/β, pAkt (Ser473) and AKT. FIG. 7F is an image analysis of the levels of p-GSK3 (Ser21/9), GSK3α/β, p-Akt (Ser473) and AKT. ***p<0.001, *p<0.05 compared with vehicle-treated TgCRND8 mice by one-way ANOVA. Values denotes group mean±SEM. N=6 mice per group.

FIG. 8 show the effects of BRR treatment on the levels of a-secretase ADAM-10, Aβ-degrading enzymes (IDE and neprilysin), phosphorylated cyclin-dependent kinase-5 (p-CDKS) and p-stress-activated protein kinase/Jun-amino-terminal kinase (JNK). FIG. 8A is representative Western blot of the levels of α-secretase ADAM-10, Aβ-degrading enzymes (IDE and neprilysin), phosphorylated cyclin-dependent kinase-5 (p-CDKS) and p-stress-activated protein kinase/Jun-amino-terminal kinase (JNK) after BRR treatment. FIG. 8B is a densitometric analysis of ADAM-10, IDE, neprilysin, p-CDKS and p-JNK shows that there is no significant difference between vehicle and BBR treatments.

FIG. 9 show protein levels of APP, CTFs, p-APP, p-CTFs, PHF-1 and p-GSK3α/β in WT and TgCRND8 mice. FIG. 9A is western blots of various protein levels in WT and TgCRND8 mice. FIG. 9B is a densitometric analysis of western blot and the protein levels were compared between APP TgCRDN8 mice (n=3) and wild-type controls (n=3). **p<0.01, *p<0.05 compared with WT mice by student t test.

FIG. 10 show BBR reduces GSK-3 activation. BBR induces concentration- and time-dependent increases in the phosphorylation of Akt and inactive GSK3α and GSK3β expression in vitro. FIG. 10A is western blot of N2a-SweApp cell lysates treated with different concentrations of BRR for 3 hours against anti-pAkt, anti-Akt, anti-pGSK3α/β (Ser21/9) anti-GSK3 polyclonal antibody recognizes total GSK3α or β. FIG. 10B shows the ratio of pAkt to total Akt, pGSK3α to total GSK3α and pGSKβ to total GSK3β in N2a-SwApp cells having treated with different concentrations of BBR for 3 hours. FIG. 10C is western blot of N2a-SweAPP cell lysates treated with 20 μM BBR over a range of time points against anti-pAkt, anti-Akt, anti-pGSK3α/β (Ser21/9) anti-GSK3 polyclonal antibody recognizes total GSK3α or β. FIG. 10D shows the ratio of pAkt to total Akt, pGSK3α to total GSK3α and pGSK3β to total GSK3β in N2a-SwApp cells having treated with 20 μM BBR over a range of time points. (n=3 for each condition) One-way ANOVA followed by post hoc comparison showed a significant difference (*p<0.05, **p<0.01 and ***p<0.001) between specific concentration- or time-group and the control group (0 μM or 0 minutes).

FIG. 11 show treatment of N2a-SwedAPP with BBR results in the inhibition of pAPP(Thr668), CTFs and PHF-1. FIG. 11A is western blots of APP, p-APP, CTFs and PHF-1 content in N2a-SwedAPP cell lysate having treated with different concentrations of BBR for 12 hours. FIG. 11B illustrates the ratios of APP, p-APP, CTFs and PHF-1 to β-actin in N2a-SwedAPP cell lysate having treated with different concentrations of BBR for 12 hours. FIG. 11C is western blots of APP, p-APP, CTFs and PHF-1 content in N2a-SwedAPP cell lysate having treated with PI3K inhibitor LY294002 for 1 hr prior to the exposure of BBR (20 μM). FIG. 11D is a densitometry analysis of band density ratio of p-APP, CTFs and PHF-1 to β-actin. Values represent as mean±SEM from three independent experiments (n=3). One-way ANOVA revealed significant differences between BBR-treated cells and control cultures (p<0.05, **p<0.01, ***p<0.001. FIG. 11E are fluorescence microscopy images of p-APP distribution in N2a-SwdAPP cells in the endosomal compartments. After treatment for 3 hr with DMSO vehicle control (A1-A4), 20 μM BBR (B1-B4), or 20 μM BBR after 1 h pretreatment with 20 μM LY294002 (C1-C4), cells were fixed and immunostained using pAPP and Rab-5 (endosome marker) antibodies and visualized using Alexa 488 (red) and 594 (red) fluorophores respectively. Nuclei were visualized using DAPI staining. Scale bar: 5 μm. Staining was analyzed using the Softmax 4.8 software for Delta Vision imaging system.

### DETAILED DESCRIPTION OF THE INVENTION

The present invention is not to be limited in scope by any of the specific embodiments described herein. The following embodiments are presented for exemplification only.

There is a need for introducing a drug on the market that is efficient in the therapy or the prophylaxis of Alzheimer's disease. The present invention discloses the use of a known drug berberine (BBR) for treatment and prophylaxis of Alzheimer's disease.

The present invention is directed to a method of preventing or treating Alzheimer's disease in a subject by administering to said subject a composition comprising berberine (BBR), which inhibits the accumulation of amyloid-β plaque and tau phosphorylation in said subject's brain. Another embodiment of the present invention relates to the use of elucidating the mechanism of BBR in N2a cell expressing Swedish mutant APP. In one embodiment, the effective dosage of the berberine ranges from 25 to 100 mg per Kg of said subject's body weight per day. In another embodiment, the dosage is 25 mg per Kg of said subject's body weight. In yet another embodiment, said subject is a mammal. In particular, said subject is human.

BBR is an isoquinoline alkaloid compound, also known as a 9,10-dimethoxy-5,6-dihydro[1,3]dioxolo[4,5-g]isoquino[3,2-a]isoquinolin-7-ium, having structure of formula I:

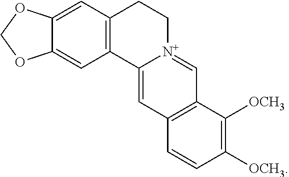 [SMILES: COc1ccc2cc3[n+](cc2c1OC)CCc1cc2c(cc1-3)OCO2]

BBR is isolated from Coptis chinensis Franch. (Huanglian, Chinese goldthread) found in China. Coptis chinensis has been used in Chinese medicine with a long history of clinical benefits. BBR is one of the important components of Oren-gedoku-to (Huanglian-Jiedu-Tang) extract, which has been used in clinical therapies for several types of dementia in China and Japan.

Animals and Treatment

All animal procedures were approved by the Hong Kong Baptist University Committee on the Use of Human and Animal Subjects in Teaching and Research and by the Committee on the Use of Live Animals for Teaching and Research (CULATR), The University of Hong Kong. TgCRND8 mice expressing human APP695 with the Swedish (K670N/M671L) and Indiana (V717F) mutations under the regulatory control of the PrP gene promoter, heterozygous with respect to the transgene, on a C57BL/6 F3 background were used to breed a colony of experimental animals. When TgCRND8 mice are three months of age, Aβ deposits become visible in their cortical brain and hippocampal regions together with astrocytic activation, microglial activation, neuritic dystrophy, inflammation and behavioral deficits that closely resembles human AD.

All animals were housed 4-5 to a cage, of the same gender, and maintained on ad libitum food and water with a 12-hour light/dark cycle in a controlled environment. Berberine (BBR) hydrochloride (98% purity) was purchased Sigma-Alridch (St. Louis, Mo.). BBR administration started at 2 months of age and lasted for 4 months up to 6 months of age. Tg mice were orally administered by gavage once daily with a low dose of BBR (25 mg/Kg/d), a high dose of BBR (100 mg/Kg/d), or vehicle for 6 months before being killed. The body weight, coat characteristics and in-cage behavior were monitored throughout the study.

Morris Water Maze

After 4 months of drug treatment, mice were assessed for spatial reference memory in the Morris water maze (MWM). The water maze consists of a white circular pool of a diameter of 100 cm and filled with opaque water (22±1° C.). A white plexiglass platform (9 cm diameter and 29 cm height) was submerged in one of the pool quadrants. The pool was virtually separated into 4 quadrants and north, west, south and east positions located at the intersections of the quadrants. The visible platform trails were used to evaluate sensorimotor and/or motivational deficits that could influence performance during the spatial navigation task. Mice underwent visible-platform training for two consecutive days (4 trials/day), each time with the platform in a different location; mice were allowed to swim to a flag-mounted platform (length 10 cm) located above the water. Training on the hidden platform water maze task commenced 24 h after the last visible-platform trial. Hidden-platform training was carried on over 6 consecutive days until each mouse had reached the criterion. During the hidden-platform trial, a set of distal visual cues was used on the black screen around the pool. The hidden platform was submerged 1 cm below the surface of the water in the southwest quadrant of the pool (target quadrant) and invisible to the mice while swimming Mice were permitted a maximum time of 60 s, starting from release in a randomly chosen quadrant to find the hidden platform. On each testing day, animals performed four trials separated by a 30 min interval.

To assess memory retention, a probe trial was performed at the beginning of the 3^{rd}, 5^{th} and 24th hour after the last training trial. In this trial, the platform was removed from the pool and the mice were permitted to swim freely for 60 s to search for the platform. The time spent in the target quadrant was taken to indicate the level of memory retention that had taken place after learning. During each trial, the distance taken to find the hidden platform (Path length in cm) and percent time spent in each quadrant of the pool during probe trials of the mouse were recorded using a video-tracking system (EthoVision 2.0, Noldus Information Technology, Leesburg, Va.).

N2a-SwedAPP Cell Culture and BBR Treatment

N2a-SweAPP cells are mouse neuroblastoma N2a cells stably transfected with human Swedish mutant APP695, and were gifts from Dr. Gopal Thinakaran (University of Chicago, Chicago Ill.). Cells were maintained in Dulbecco's modified Eagle's medium (DMEM) (Invitrogen, Carlsbad, Calif.) supplemented with 10% fetal bovine serum, penicillin and streptomycin and 200 μg/mL G418 (Invitrogen). Cells were cultured either in multiwell dishes or on 60 mm tissue culture plates in DMEM 10% with FBS Cell cultures were incubated at 37° C. in a humid 5% CO₂/ 95% air environment. After cells were grown close to confluence, they were treated with/without different concentrations of BBR in DMEM (1% FBS) for 24 h. We first measured whether BBR showed toxicity in the N2a-SweAPP cells using the 3-(4, 5)-dimethylthiahiazo(-z-yl)-3,5-diphenytetrazoliumromide (MTT) (Sigma-Aldrich, St. Louis, Mo.) assay. The treatment of BBR did not affect cell viability up to 25 μM in N2a-SwedAPP cells. For the concentration-dependent treatment, cells were treated with serial dilutions of BBR ranging from 0-20 μM for 2 or 12 h prior to lysis with ice-cold RIPA buffer (20 mM Tris, pH 7.4, 150 mM NaCl, 1% triton-X, 0.5% sodium deoxycholate, EDTA) containing protease inhibitor cocktails (EMD chemicals, Philadelphia, Pa.) to find the optimal range of BBR. For the time-dependent application, cells were treated with a 20 μM concentration of BBR and incubated at time points ranging from 0-180 min prior to lysis. For immunofluorescent staining, N2a-SwedAPP cells were seeded on glass cover slips at a density of 0.5×10⁵ viable cells per well in a 24-well plate. Cells on cover slips were treated with BBR (20 μM) for 3 h with or without pretreatment of LY294002 (Cell signaling Technology, Danvers, Mass.), a specific inhibitor of Akt upstream kinase phosphatidylinositol 3-kinase (PI3K) (Vlahos et al., 1994) in serum-free DMEM for 1 h, whereas control cells were incubated with vehicle (DMSO) alone in DMEM.

Immunohistochemistry and Immunocytochemistry

After the Morris water maze experiment, animals were deeply anesthetized with an intraperitoneal injection of and transcardially perfused with 0.9% saline. The brains were removed and bisected in the mid-sagittal plane. Half of each brain was frozen on dry ice for Aβ ELISA analysis. One hemisphere was fixed in 4% formaldehyde for 48 h and transferred to 30% sucrose/PBS for immunohistochemistry. Three coronal sections (per set) from anterior, medial, and posterior hippocampus with a 120-μm interval were made on a cryostat at −20° C. at a thickness of 30 μm in each region. Three sets of sections in each region were prepared for analyses of Aβ, Iba-1 (ionized calcium binding adaptor molecule 1) (microgliosis) and glial fibrillary acidic protein (GFAP) (astrocytosis). Sections were immunostained using the following antibodies: a biotinylated human amyloid-β monoclonal antibody (4G8; 1:1000; Covance, Princeton, N.J.), GFAP polyclonal antibody (1:500; DAKO, Carpinteria, Calif.), and an Iba-1 polyclonal antibody (1:1000; Wako, Osaka, Japan) Immunohistochemical staining was performed according to the manufacturer's instructions using a Vectastain ABC Elite kit (Vector Laboratories, Burlingame, Calif.) linked with the diaminobenzidine reaction, with the exception that the biotinylated secondary antibody step was excluded for Aβ staining. Isotype control serum or phosphate-buffered saline (PBS, 0.1 mM, pH 7.4) was used instead of primary antibody as a negative control. All images were acquired using a Nikon fluorescent inverted microscope with image acquisition system. A threshold optical density representing specific immunoreactive signal was established after subtracting the background and nonspecific staining level, and was then held constant throughout the image analysis. To facilitate quantitative measurement of Aβ burden, a region of interest was captured on each section from anterior, medial, and posterior regions (9 sections per mouse). Images were converted to gray scale and thresholded using an unbiased computer assisted Image J software (NIH). Quantification of Aβ immunostaining was performed to detect the proportion of region occupied by Aβ immunoreactivity. Aβ burden expressed as the area of Aβ per the total area of the region-of-interest. Iba-1 (microgliosis) and GFAP (astrocytosis) burden was also estimated as the area of immunoreactivity expressed as a percentage of full area. For Aβ plaque morphometric analyses, diameters of plaques were measured, and numbers of plaques in three categories of diameter (25, 25-50, or >50 μm) were calculated. Assessments were carried out by a single examiner blinded to sample identities.

For immunofluorescent staining, cells were fixed, permeabilised, blocked and stained with a combination of primary antibodies. To allow combination with the first and second primary antibodies, both raised in the same species, further immunostaining was done following the method reported by Negoescu, A., Labat-Moleur, F., Lorimier, P., Lamarq, L., Guillermet, C., Chambaz, E., Brambilla, E., 1994. F(ab) secondary antibodies: a general method for double immunolabeling with primary antisera from the same species. Efficiency control by chemiluminescence. J. Histochem. Cytochem. 42: 433-437 with little modifications. Permeabilised cells were incubated with primary antibody specific for phosphorylated APPThr668 (p-APP) (1:100, Cell Signaling Technology) overnight at 4 C. After incubation of the first primary antibody, cells were rinsed with PBS containing 0.05% Tween-20 (PBST) three times. Subsequently, cell were incubated with Alexa Fluor® 488-conjugated Fab fragments of goat anti-rabbit immunoglobulin (Ig)G (H+L) for 1 hr (1:500, Invitogen). After washing, cells were incubated with unlabeled Fab fragments of goat anti-rabbit IgG (H+L) for 1 hr (1:100, Invitrogen) to block all possible remaining binding sites of the second primary antibody. The p-APP-labeled cells were incubated with a second rabbit polyclonal antibody raised against Rab5 (endosome marker) (1:100, Cell Signaling Technology) overnight, followed by an Alexa Fluor® 594-conjugated Fab fragment of goat anti-rabbit IgG (H+L) (diluted 1:500), for 1 hr. After several washes with PBST, cells were mounted using an anti-fading mounting medium.

For higher resolution microscopy, images were acquired using an Olympus IX70 Delta Vision microscopy system (Applied Precision, Issaquah, Wash.) with a 100×1.5 numerical aperture oil immersion lens. Images were collected using a Cool SNAP digital camera (Photometrics, Tucson, Ariz.). Subsequently serial Z-stacks of fluorescence images at 0.2-μm interval were acquired and deconvolved via a controlled iterative algorithm to generate high-resolution images of cells using Softmax software (Applied Precision).

Measurements of Aβ1-40 and Aβ1-42 in Brain Extracts

To detect differences in the level of Aβ among treatment groups, the levels of Aβ were measured using the two-step sequential extraction of SDS and formic acid (FA) methods. Briefly, each frozen hemisphere was first homogenized in 2% SDS in water containing protease inhibitors (Complete mini Protease Inhibitor Cocktail; Roche Diagnostics, Basel, Switzerland) and phosphotase inhibitor (EMD chemicals). After sonication, samples were centrifuged at 100,000 g at 4° C. for 1 h to obtain a soluble SDS fraction, which was then stored at −80° C. The resulting SDS pellets were resuspended in a 70% FA and centrifuged at 100,000 g at 4° C. for 1 h. Supernatants were collected and stored at −80° C. until used to measure insoluble Aβ. The 2% SDS extracts were diluted at least 1:300 in 1% BSA in PBS so that the assays were performed in 0.006% SDS. The FA extract was neutralized by a 1:20 dilution into 1M Tris, pH 11. The neutralized FA extracts were diluted 1:2 in 1% BSA in PBS. Brain extracts were measured for Aβ(1-40) and Aβ(1-42) using ELISA. Absorbance values at 450 nm were measured in duplicate wells, and the average of the signal from the two wells was considered to represent the Aβ concentration for the sample.

Western Blot Analyses

The brain SDS fraction as described above was used to detect the full-length APP (Fl-APP), APP C-terminal fragments (CTFs: CTF-β and CTF-α), p-APP, p-CTFs, BACE-1, ADAM-10, neprilysin, insulin-degrading enzyme (IDE), GSK3α/β, p-cyclin-dependent kinase 5 (p-CDKS), p-stress-activated protein kinase/Jun-amino-terminal kinase (p-JNK), phospho-tau epitopes and β-actin in Western blot analysis. For cell homogenates preparation, cells were washed with phosphate-buffered saline (PBS) and solubilized in ice-cold RIPA buffer. Equal amounts of protein (30 μg) were subjected to 10 and 15% SDS polyacrylamide gels, transferred onto PVDF membranes (GE Healthcare, Piscataway, N.J.) at 300 mA for 2 h, and then blocked with 5% fat-free milk in Tris-buffered saline and 0.1% Tween-20 for 2 h at room temperature. The membrane was then incubated with primary antibodies overnight at 4C. The antibodies used in Western blots were β-actin (mouse, 1:5000; Santa Cruz, Calif.), p-APP (rabbit, 1:1000; Cell signaling Technology), BACE-1 (rabbit, 1:1000; Abcam, Cambridge, Mass.), ADAM-10 (rabbit, 1:2000; EMD chemicals), insulin-degrading enzyme (IDE) (rabbit, 1:1000, Abcam), neprilysin (rabbit, 1:1000 Millipore, Temecula, Calif.), Akt (rabbit, 1:1000; Cell Signaling Technology), p-Akt (rabbit, 1:1000; Cell signaling), glycogen synthase kinase 3α/β(GSK-3α/β)[Ser21/9] (rabbit, 1:1000; Cell signaling Technology), pGSK-3α/βSer21/9] (rabbit, 1:1000; Cell signaling Technology), Cdk5[Y15] (rabbit, 1:1000; Abcam), JNK (Thr183/Tyr185) (rabbit, 1:1000, Cell signaling Technology), the C-terminal anti-APP antibody CT15 for full-length APP and CTFβ and CTFα (rabbit, 1:2000, and three mouse monoclonal antibodies against phosphorylated tau: AT8 (recognizes phosphorylated tau at Ser-202 and Thr-205, 1:1000, Pierce, Rockford, Ill.), AT180 (recognizes phosphorylated tau at Thr-231 and Ser-235, 1:1000, Pierce) and PHF-1 (recognizes -phosphorylated tau at Ser-394 and Ser 404, 1:2000, and one mouse monoclonal against dephosphorylaed tau: Tau-1 (recognizes the tau when serines 195, 198, 199, and 202 are dephosphorylated, 1:2000,). Mouse monoclonal Tau-5 used to recognize total tau (mouse 1:2000). Blots were washed in and incubated with corresponding horseradish peroxidase-conjugated secondary antibody. Secondary antibody was HRP-conjugated IgG anti-mouse (goat anti-mouse, 1:5000; Invitrogen) or anti-rabbit (goat anti-rabbit, 1:10,000; Invitrogen) as needed. Supersignal chemiluminescent substrate (Pierce, Rockford, Ill.) was used to visualize HRP activity on Fuji films (GE Healthcare). The omission of primary antibody resulted in negative staining Immunoblots were quantified using NIH Image J software, with optical density (OD) measures adjusted for individual loading control OD levels.

Statistical Analysis

Behavioral data were analyzed with two-way analysis of variance (ANOVA) for repeated measures with ‘treatment, and ‘day’ and their interactions as fixed factors. After ANOVA analysis, post hoc pair-by-pair differences between groups were determined using the Fisher LSD test. Immunohistochemical data were also analyzed using a one-way and two-way ANOVA performed with a Fisher LSD-post hoc comparison. Calculations and graphical presentation were performed with the statistical software GraphPad Prism 5 (GraphPad Software, San Diego, Calif.) Software Inc., Chicago, Ill., USA) and Sigmaplot™ (Version 10, SPSS, Chicago, Ill.). Results are presented as mean±S.E.M

Results

Regular Treatment and Levels of BBR in TgCRND8 Mouse Brain

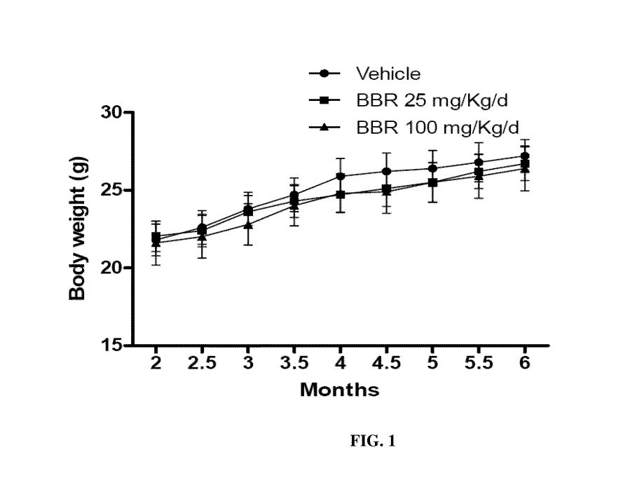

Regular treatment of TgCRND8 mice with BBR orally administered at 25 or 100 mg/kg/d for 4 months, does not significantly influence animal body weight nor does it cause any noteworthy adverse side effects (FIG. 1). To determine how oral administration of BBR influences the concentration of BBR in the brain, liquid chromatography and mass spectrometry techniques (LC/MS) are used to analyze changes in BBR levels in the TgCRND8 brain tissue in a separate experiment. BBR levels in the brain are found to be 0.88±0.27 and 1.67±0.73 μM for low and high doses of BBR respectively after 3 hr p.o. administration.

BBR Treatment Reverses Deficits of Learning and Memory in TgCRND8 Mice

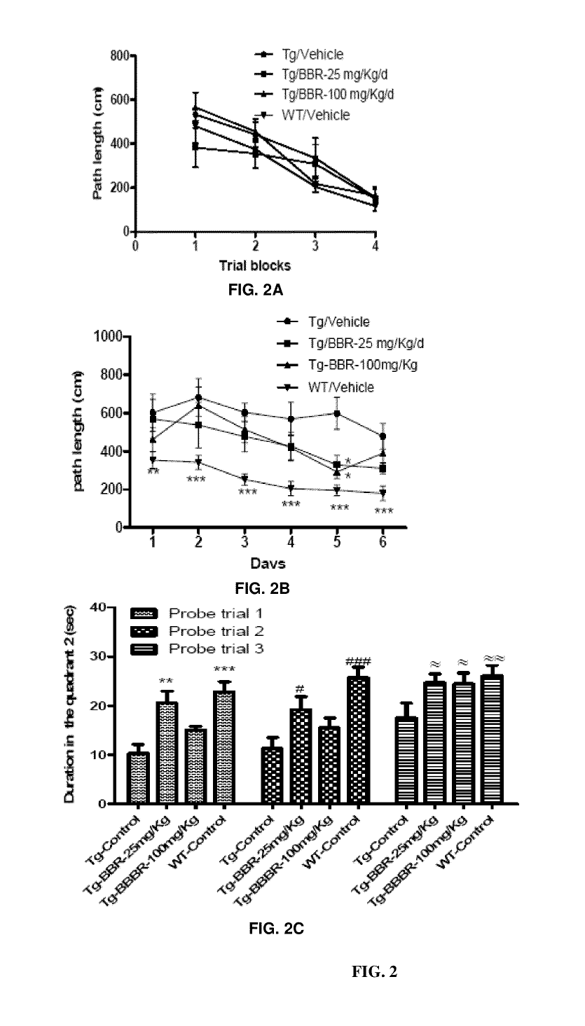

At the beginning of the fifteenth week of BBR treatment, both Tg and WT mice were trained for four days (four trials/per day) in the MWM with the visible platform so that they could learn where the platform was located. Although distance traveled for BBR-treated TgCRND8 mice on the first training day of the visible water maze tends to be lower than distances for vehicle treated TgCRND8 mice, differences in overall performance are insignificant (p=0.055) (FIG. 2A). The visible platform tests show that the BBR group and Tg control group has a similar path length from 2 to 4 days as revealed by posthoc Fisher LSD multiple comparisons tests (p>0.05; FIG. 2A), demonstrating that BBR treatment does not significantly affect motility or vision in the BBR-treated mice. In other words, the BBR treatment does not influence the visible platform learning trend, which is used as control for the non-spatial factors (e.g., sensory-motor performance).

After the visible platform training, both Tg and WT mice were trained for six days in the MWM so that they could learn where the hidden platform was located. From the 24 training trials, average measures of total distance to the platform for each 4-trial block were analyzed to determine and distinguish genotype and treatment differences in spatial learning. Vehicle-treated TgCRND8 mice exhibit a longer path distance [F_{(1.96)}=108.6, p<0.001[) when compared with the vehicle-treated WT mice during all trial sessions (FIG. 2C). Treatment with low and high doses of BBR reduced the path distance in TgCRND8 mice compared with placebo-treated TgCRND8 mice [FIGS. 2B and 2C; F_{(2,108)}=7.6, p<0.001, respectively]. FIG. 2C also shows that the placebo-treated TgCRND8 mice experienced a spatial learning impairment compared to the WT controls and BBR-treated groups.

In post hoc multiple comparisons, all Tg groups show no significant changes in swimming distance during the first day (p>0.05). When the vehicle-treated TgCRND8 control group is compared with WT controls, vehicle-treated TgCRND8 mice show significantly longer swimming distances than WT-control group mice, from the 1^{st} to 6^{th} day (p<0.001). From days 4 to 5, TgCRND8 mice treated with 25 and 100 mg kg d⁻¹ of BBR show a reduction in swimming distance as compared with vehicle control group mice, reaching similar values to those of WT-control group (FIG. 2B).

MWM behavior testing confirms that both of the two different doses of BBR (25 and 100 mg/kg/d) not only significantly enhanced learning skills during the hidden-platform learning trial, but also significantly enhanced spatial memory retention during the probe trial. The spatial bias of the mice for the position of the hidden escape platform is revealed by the duration spent by the mice in the quadrant of the maze that retained the platform throughout the three interpolated probe trials (FIG. 2C). Analyses of probe test search ratio data indicate a significant effect of treatment [F_{(3,81)}=16.68, p<0.001] and a significant effect of trial [F_{(3,81)}=8.17, p<0.001]. Posthoc Fisher LSD multiple comparison tests indicate that the TgCRND8 mice in the BBR 25 mg/Kg treatment group significantly increases their spatial bias in all the probe trials (post hoc Fisher's LSD). Vehicle-treated mice show lower times in the target quadrant (p<0.001) and lower numbers of crossings than vehicle-treated WT mice. Both doses of BBR (25 and 100 mg/kg/d) prevent decrease in the time spent in the target quadrant (p<0.001) of the previous location of the platform (FIG. 2C). These results demonstrate that spatial memory impairment in vehicle-treated TgCRND8 mice is alleviated by prolonged treatment with BBR.

If the animals had spent more time and had swum further in the target quadrant where the platform had previously been placed during the training session, this would have indicated that the animals had acquired spatial memory improvement. Therefore, these results suggest that BBR can ameliorate the long-term memory loss in TgCRND8 mice.

BBR Mitigates Aβ Pathology and Aβ Levels in TgCRND8 Mice

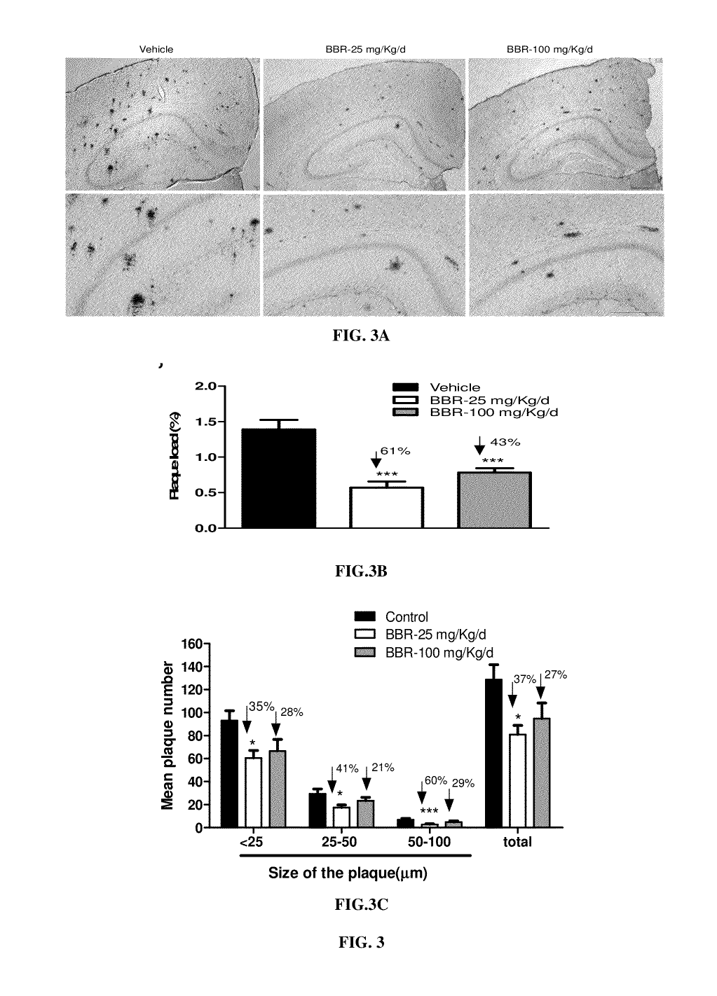

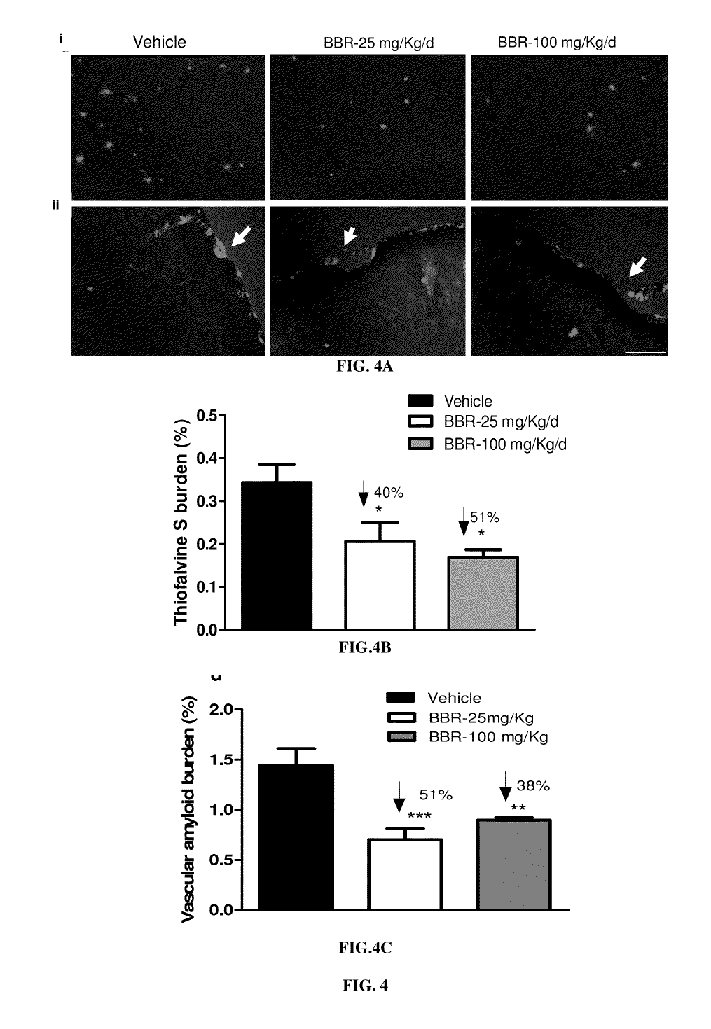

The observation that BBR treatment can alleviate the learning and memory deficit led to further examination if there is any correlation in the reduction of Aβ plaque deposition by BBR in TgCRND8 mice. Aβ plaque immunostaining with the 4G8 antibody and ThioS staining in TgCRND8 mice disclose marked Aβ deposits both in the cerebral parenchyma and in cerebral blood vessels at 6 months of age. Examination of amyloid plaque deposition in the brains of TgCRND8 mice reveals that BBR treatment shows significant reduction in the number and area occupied by Aβ deposits in the coronal sections of the cortex and hippocampus (FIG. 3A). Quantification of the immunoreactivity shows significantly lower plaque burden in BBR-treated animals compared with controls. BBR treatment at concentration of 25 mg/Kg/d resulted in a 61% (p<0.001) decrease in plaque burden compared with control animals, whereas 100 mg/Kg/d of BBR treatment reduces Aβ plaque burden to 43% of untreated control levels (p<0.05) (FIG. 3B).

To further measure which subsets of Aβ plaques are reduced, morphometric analyses of Aβ plaques are performed on whole brain. The two-way ANOVA revealed both a treatment [F_{(2,68)}=17.4, p<0.001] and a size effect [F_{(3,68)}=292.6, p<0.001] in the mean number of Aβ plaques. Posthoc multiple analysis reveals that BBR (25 mg/Kg)-treated TgCRND8 mice shows a significant reduction in large (>50), medium (25-50 μm), small sized (<25 μm) and total amyloid plaque subsets (percentage reduction in large, medium, small and total subsets are 60% (p<0.001), 41% (p<0.05), 35% (p<0.055) and 37% (p<0.05), respectively (FIG. 3C). 100 mg/Kg/d of BBR dose produces a 21-28% reduction of amyloid plaque subsets (FIG. 3C).

Moreover, consistent with the indication that BBR treatment reduces the accumulation of total Aβ peptides in the brain a significant reduction in ThioS-positive fibrillar Aβ in the brains of the same TgCRND8 mice at 25 mg/kg/d (40%, p<0.05) or 100 mg/kg/d (51%, p<0.05), relative to age-matched untreated control TgCRND8 mice, as examined stereologically is also found (FIGS. 4A and 4C). It has been reported in Hawkes C. A., McLaurin J. Selective targeting of perivascular macrophages for clearance of beta-amyloid in cerebral amyloid angiopathy. Proc Natl Acad Sci U S A. 2009;106:1261-1266; Cortes-Canteli M, Paul J, Norris E H, Bronstein R, Ahn H J, Zamolodchikov D, Bhuvanendran S, Fenz K M, Strickland S. Fibrinogen and beta-amyloid association alters thrombosis and fibrinolysis: a possible contributing factor to Alzheimer's disease. Neuron. 2010 66:695-709 that TgCRND8 mice are more prone to vascular amyloid deposition in leptomeningeal and cortical blood vessels resembling human cerebral amyloid angiopathy (CAA) symptoms. To determine the effect of BBR on cortical CAA severity, brain sections were stained with ThioS to detect fibrillar Aβ. It is found that CAA-immunolabeled areas occupied 1-1.5% in each region examined in vehicle-treated mice (FIG. 4B), and treatment with BBR showed a significant decrease in the intensity of staining and the number of ThioS-labeled blood vessels compared with vehicle-treated mice (vehicle, 1.44±0.17% cortical area covered; 25 mg/Kg/d of BBR 0.70% ±0.11% (p<0.001); 100 mg/Kg/d of BBR 0.89%±0.02% (p<0.05) (FIGS. 4B and 4C)

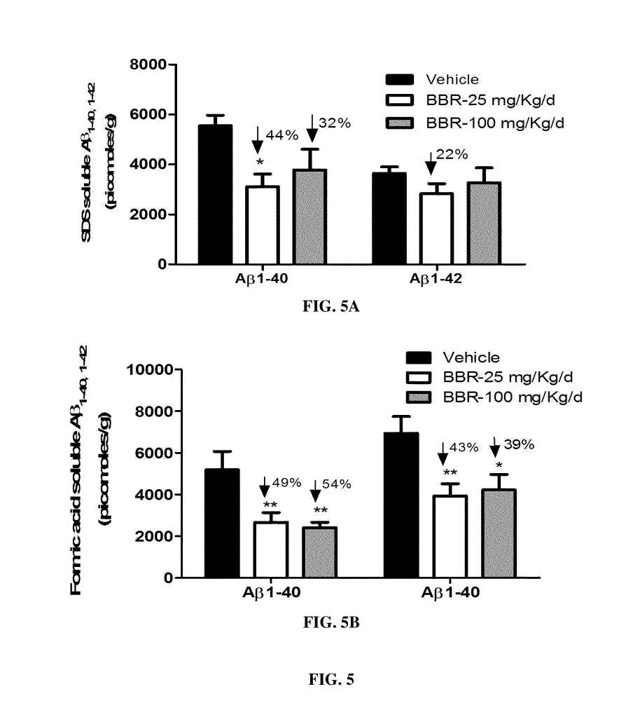

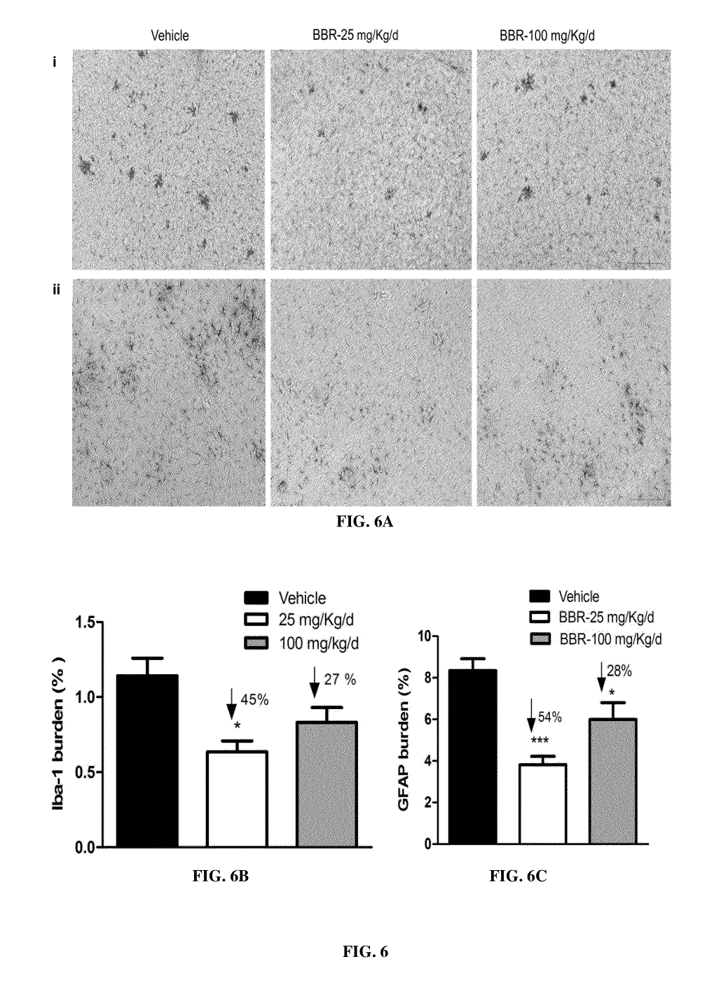

The above findings of reduced 4G8-positive and ThioS Aβ deposits are further corroborated by Aβ ELISA analysis in the opposite hemisphere of brain. In particular, this assay discloses that Aβ levels in both SDS-soluble and insoluble formic acid fractions are significantly decreased in BBR-treated TgCRND8 mice (FIGS. 5A and 5B). Soluble Aβ was first extracted with 2% SDS, and the remaining Aβ was pelleted at 100,000×g and extracted with 70% formic acid. In the SDS-soluble fraction, the diminution of Aβ1-40 is 44% (p=0.011) and 32% (p=0.051) by 25 mg/Kg/d and 100 mg/Kg/d, respectively, and there is slight reduction of Aβ1-42 by BBR treatments (FIG. 5A). One-way ANOVA analyses of both Aβ1-40 and Aβ1-42 in the formic acid fraction shows treatment effects from both low and high concentrations of BBR. In the formic acid extractable fraction, the reductions of Aβ1-40 are 49% (p<0.01) and 54% (p<0.01) (FIG. 6b), and the corresponding reductions of Aβ1-42 are 44% (p<0.01) and 39% (p<0.05) by 25 mg/Kg/d and 100 mg/Kg/d of BBR, respectively.

BBR Ameliorates Aβ-Associated Reactive Gliosis and Astrocytosis in TgCRND8 Mice

Microgliosis and astrocytosis in TgCRND8 mice are investigated which are elevated phenotypically as a consequence of amyloid deposition. Double immunohistochemical staining with Iba-1/GFAP and ThioS confirms that only activated microglial cells are in very close association with compact plaques and absent in regions lacking such deposits (data not shown). Iba-1 is expressed in microglia/macrophages, and is upregulated during activation of these cells. The degree of microgliosis as evaluated by Iba-1 load in the three brain regions examined is significantly amplified in vehicle-treated TgCRND8 mice relative to WT mice (data not shown), whereas it is significantly reduced to 45% (p<0.05) and 27% (p>0.05) in 25 and 100 mg/Kg/d of BBR-treated TgCRND8 mice relative to vehicle-treated TgCRDN8 mice (FIGS. 6Ai and 6B). Likewise, the magnitude of astrocytosis as assessed by clusters of GFAP-immunoreactive astrocytes (GFAP burden) is significantly reduced to 54% (p<0.001) and 28% (p<0.05) in 25 and 100 mg/Kg of BBR-treated TgCRND8 mice, respectively, relative to vehicle-treated TgCRND8 mice (p<0.001) (FIGS. 6Aii and 6C). Activated microglial cells are typically associated with amyloid plaques; thus the decrease in the Iba-1 burden correlates with reduced plaque burden.

BBR Alters APP Processing, APP and Tau Phosphorylation Probably Through Inhibition of Akt/GSK3 Activities in TgCRND8 Mouse Brain

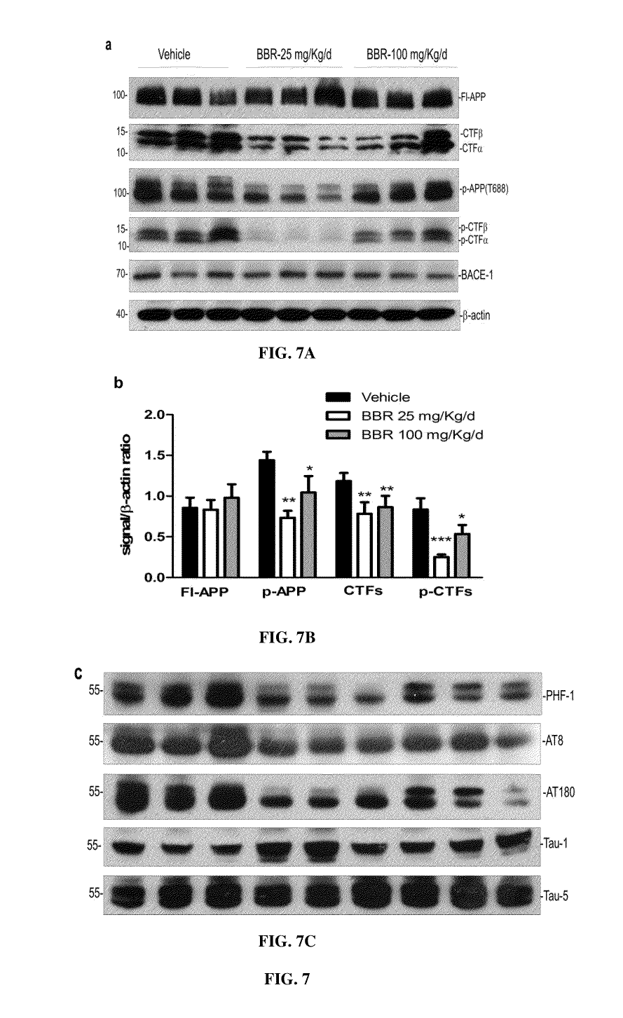

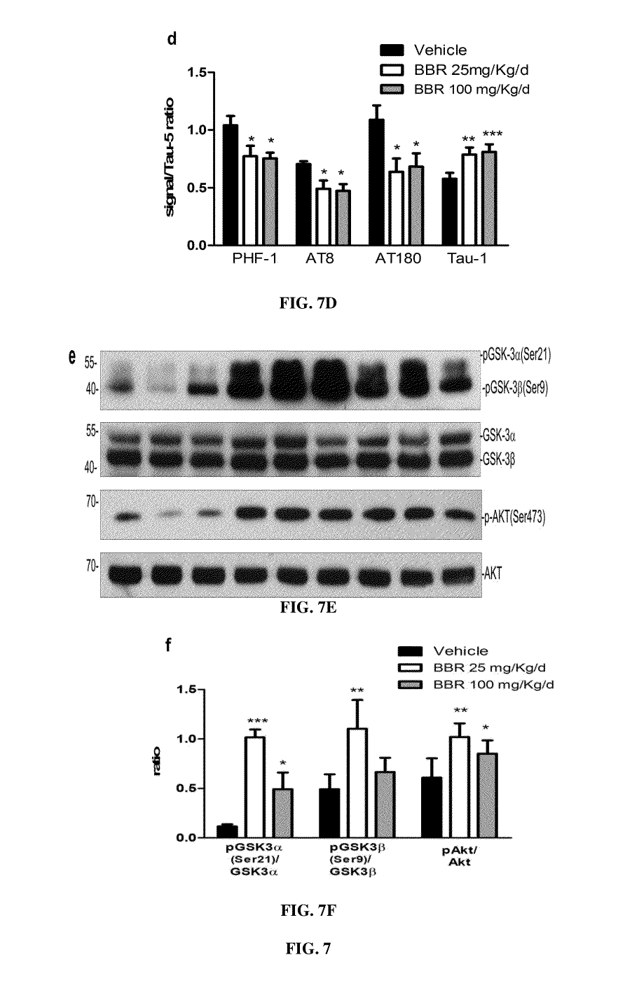

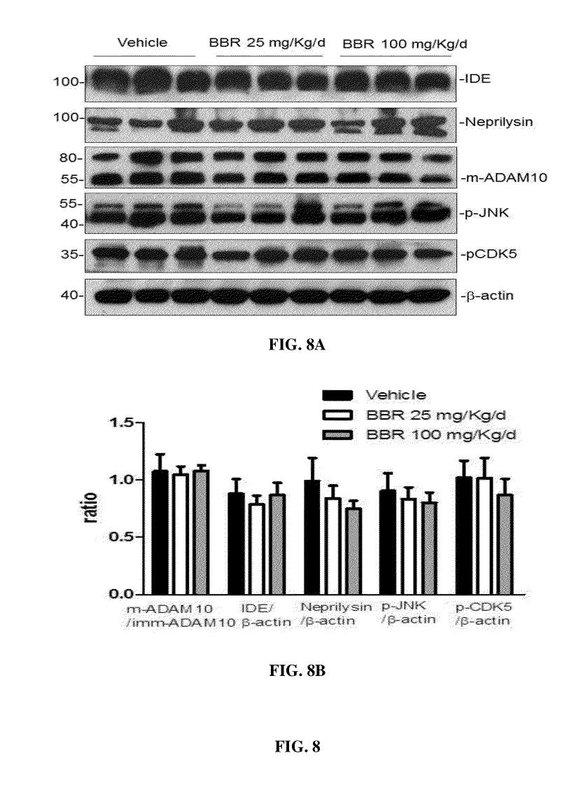

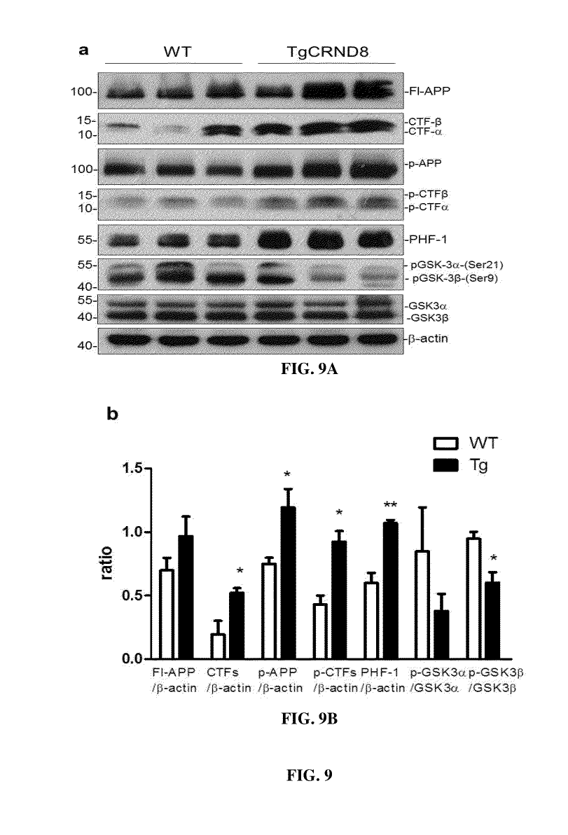

To understand the mechanisms underlying the benefits of BBR for cognitive function and Aβ neuropathology of TgCRND8 mouse, the effects of BBR on the processing of APP is examined. It is found that BBR treatment has no effect on full length APP, BACE-1 and ADAM-10 in brain homogenates (FIGS. 7A, 7B and 8). Moreover, BBR does not affect the levels of the Aβ-degrading enzymes neprilysin and IDE (FIG. 8). While BBR shows no influence on secretases, BBR treatment (25 mg/Kg/day) significantly reduces both CTF-α and β (FIGS. 7A and 7B). Taken together, these findings confirm that BBR's Aβ reducing effects might be related to regulation of APP-CTFs expression or intracellular maturation and distribution, rather than modulation of APP processing enzymes or Aβ degradation.

Several studies suggest that APP maturation, subcellular distribution and generation of Aβ are phosphorylation-dependent. It is also suggested in Lee, M. S., Kao, S. C., Lemere, C. A., Xia, W., Tseng, H. C., Zhou, Y., Neve, R., Ahlijanian, M. K., Tsai, L. H., 2003a. APP processing is regulated by cytoplasmic phosphorylation. J. Cell Biol. 163, 83-95 that APP phosphorylation modulates its metabolism, resulting in increased production of carboxy-terminal fragments (CTFs). Therefore, to ascertain levels of APP phosphorylation in the brain lysates by immunoblot analysis an antibody against p-APP-threonine668 is used. Results show that levels of p-APP and p-CTF are lower in BBR-treated mice compared with the vehicle-treated TgCRND8 mice (FIG. 7A). Robust elevation of phosphorylated APP and CTFs are detected in vehicle-treated TgCRND8 mice. In contrast, the APP and CTFs phosphorylation are significantly reduced in BBR-treated TgCRND8 mice. Quantitative analysis of immunoblots of p-APP and p-CTFs shows 46-50% decrease in the p-APP and p-CTFs in the low dose group, and 27-28% decrease in the p-APP and p-CTFs in the high dose group of the BBR-treated TgCRND8 mice compared with the vehicle (FIG. 7B).

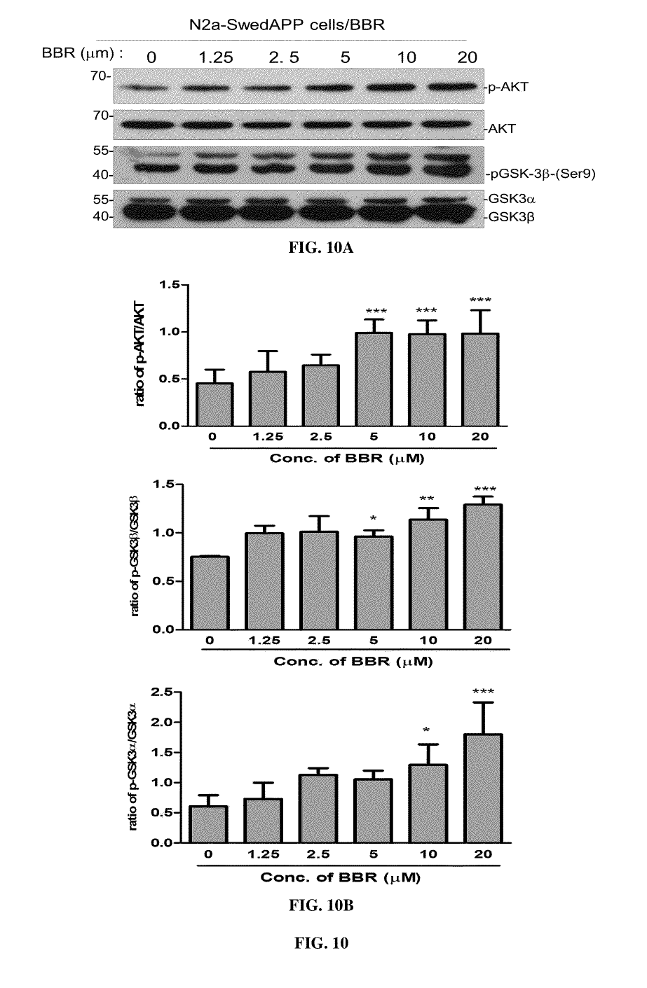

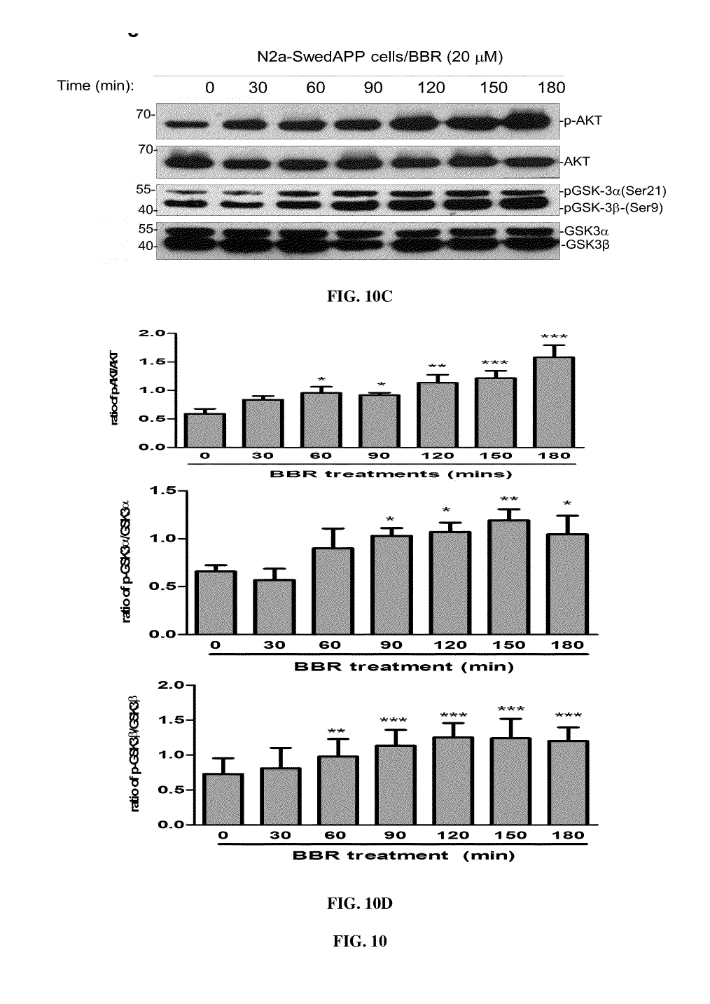

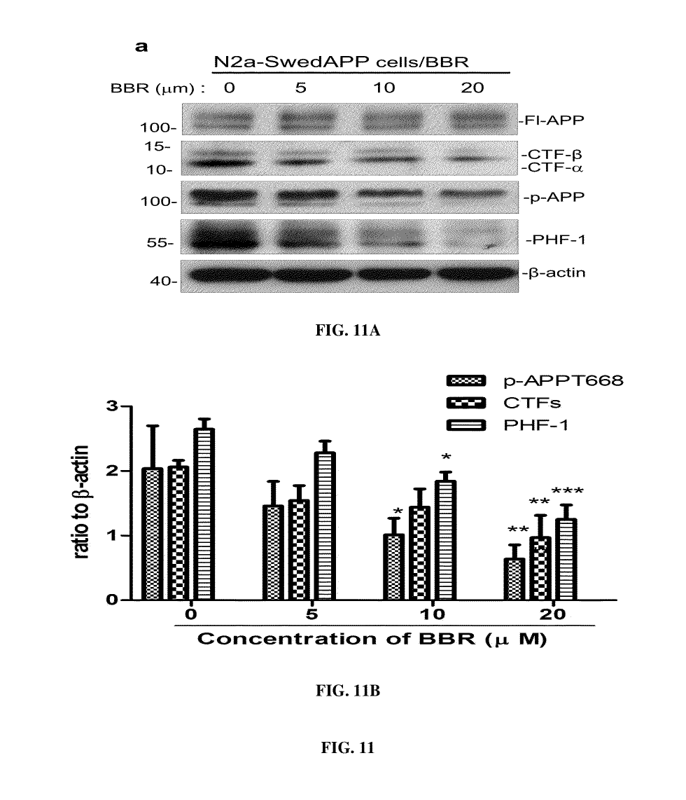

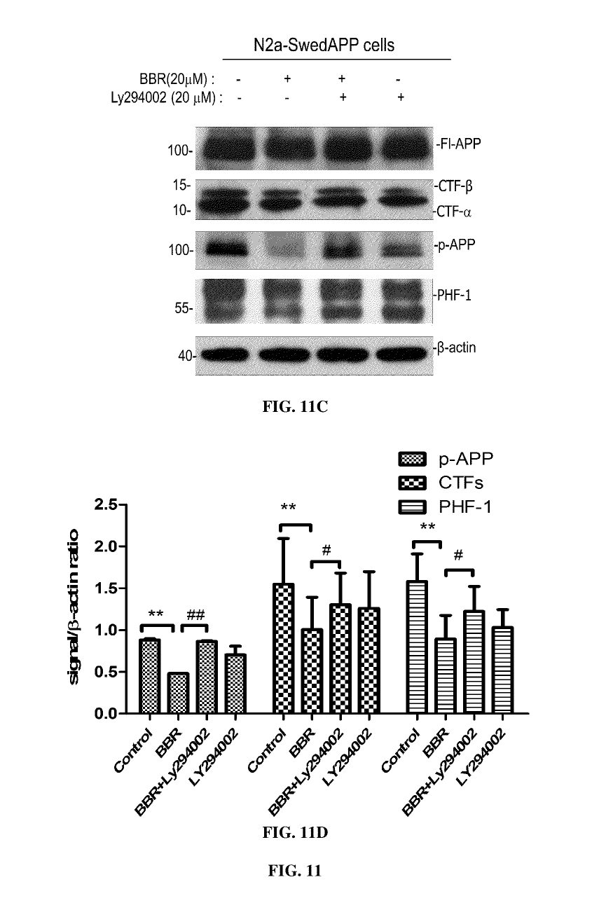

Taking into account the inhibitory role of BBR in APP phosphorylation and Aβ accumulation, a possible role of BBR in tau hyperphosphorylation in TgCRND8 mice is determined. It has been shown in Bellucci, A., Rosi, M. C., Grossi, C., Fiorentini, A., Luccarini, I., Casamenti, F., 2007. Abnormal processing of tau in the brain of aged TgCRND8 mice. Neurobiol. Dis. 27, 328-338 that amyloid accumulation in TgCRND8 mice is followed by hyperphosphorylation of tau at different sites recognized by PHF-1, AT100, AT8 and CP13 antibodies. To evaluate quantitatively the effects of BBR on tau hyperphosphorylation, total tau and phosphorylation epitopes on tau, including PHF-1, AT8 and AT180, by Western blotting are assessed. A robust reduction in the phosphotau recognized by PHF-1, AT8 and AT180 in the brain homogenates of BBR-treated TgCRND8 mice is observed (FIG. 7C). This finding is based on earlier work in Cho, J. H., Johnson, G. V., 2003. Glycogen synthase kinase 3beta phosphorylates tau at both primed and unprimed sites. Differential impact on microtubule binding. J. Biol. Chem. 278, 187-193 demonstrating that these sites are phosphorylated by GSK-3β. Quantitative analysis of the Western blot bands of the phosphorylated tau show 26%, 30% and 42% decreases at PHF-1, AT8 and AT180 phosphoepitopes, respectively, in the BBR-treated TgCRND8 mice relative to vehicle-treated TgCRND8 mice (FIG. 7D). In addition, Western blotting with a tau-1 antibody, recognizing the dephosphorylated tau at Ser198/Ser199/Ser202, show a significant increase in tau-1 immunoreactivity in BBR-treated TgCRND8 mice. No significant difference in the total tau levels is observed between the groups (FIGS. 7C and 7D). Both APP and tau hyperphosphorylation significantly increase in vehicle-treated TgCRND8 mice compared with the WT controls (FIG. 10).

Cyclin-dependent kinase 5 (CdkS), glycogen synthase kinase-3 (GSK3) and c-jun-N-terminal kinase (JNK) are the three key kinases involved in the phosphorylation of APP. Since BBR reduces the accumulation of CTFs and inhibits the phosphorylation of APP and APP-CTFs, whether BBR plays a role in regulating CdkS and/or GSK3 is sought to be detemined. It is known that GSK3 is activated through the phosphorylation at Tyr216 or is inhibited when Ser9 is phosphorylated. CdkS activity is enhanced when CdkS is phosphorylated at Y15 and associates with p35, significantly increasing its kinase activity. Thus, the levels of phosphorylated CdkS, GSK3β, and JNK using specific antibodies are examined. As shown in FIGS. 7E and 7F, a profound enhancement of inactive form of GSK3β phosphorylated at Ser9 is observed in the brains of the BBR-treated TgCRND8 mice compared with vehicle-treated TgCRND8 controls. Quantitative analysis of the Western blot bands indicates a 50% enhancement in the phosphorylated GSK3α and β at Ser 21 and 9 in the BBR-treated TgCRND8 mice relative to vehicle-treated TgCRND8 mice (FIGS. 7E and 7F). These data show that BBR treatment downregulates GSK3 activity Immunoblot analysis with antibodies against the phosphorylated CdkS and JNK show no significant differences in p-CdkS (Y15) and p-JNK (Thr183/Tyr185) respectively between the BBR- and vehicle-treated TgCRND8 mice (FIG. 8). Since Akt/GSK3 pathway is found to be dysregulated in AD, the levels of inactive GSK3 and active Akt in the TgCRND8 mouse brains are evaluated. The levels of phosphorylated Akt (p-Akt) in TgCRND8 mice that received BBR is significantly high from that of the vehicle-treated TgCRND8 mice, which show a significant reduction in p-Akt levels (normalized to total Akt) (FIGS. 7E and 7F). Increased phosphorylation of Ser9 in GSK3β indicates decreased activity of GSK3β, whereas p-Akt in Ser473 indicates increased activity of Akt. The level of the active GSK3β is increased in TgCRND8 mouse brains and that BBR treatment increases the activity of protective Akt pathway. The enhanced levels of pGSK-3β and p-Akt are not ascribable to an augment in total GSK3α/β or total Akt, because the amount of total GSK3α/β and total Akt is unaltered by BBR treatment.

BBR alters APP processing, APP and tau phosphorylation through PI3K/Akt/GSK3 pathway in vitro

Given the inhibitory role of BBR in the phosphorylation of GSK3 in TgCRND8 mice brain, phosphorylation of both GSK3α and β at Ser 21 and Ser9 level in N2a-SwedAPP cells following BBR treatment is further evaluated. Since GSK3 activity is inhibited via phosphorylation at specific serine residues (Ser 9 for GSK3β and Ser21 for GSK3α) by Akt, the effect of BBR on the protein levels of phosphorylated Akt is also investigated. The amount of phosphorylated GSK3 increases during the first 30 min of BBR treatment, and remains higher than the control group at times exceeding 3 h following BBR treatment (FIGS. 10B and 10D). BBR also increases the phosphorylation of Akt at Ser 473 and GSK3 at Ser 21 and Ser9 in a concentration-dependent manner with significant increases achieved at BBR concentrations of 10 and 20 μM, but it does not significantly affect cellular total Akt and GSK3β levels (FIGS. 10A and 10C).

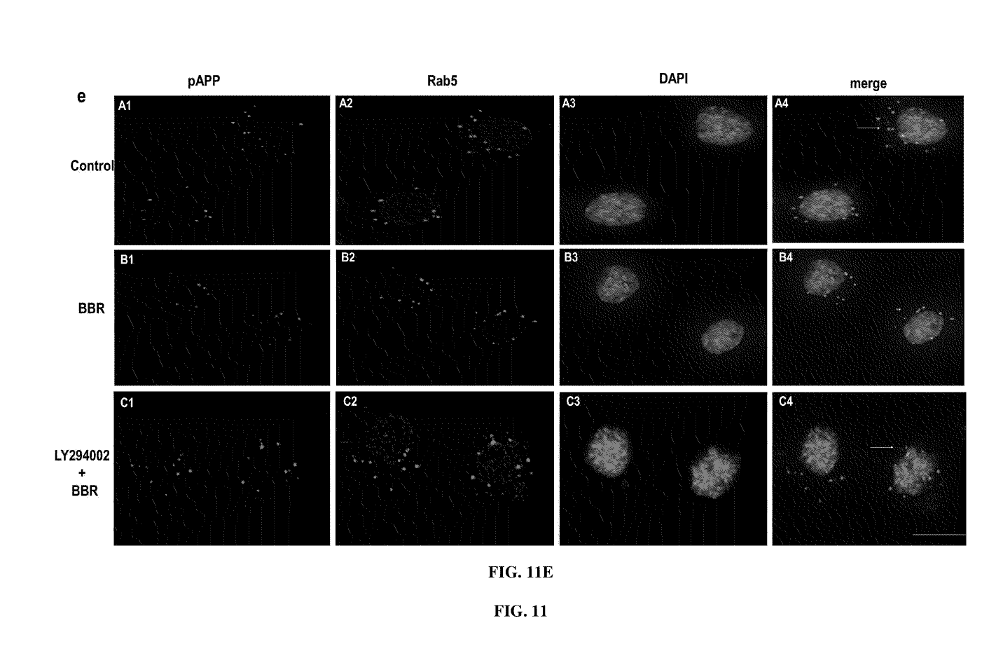

To study the effect of BBR on APP processing and tau phosphorylation in N2a-SwedAPP cells, the levels of CTFs, p-APP and PHF-1 in cell lysates is examined by western blotting analysis. As shown in FIG. 11A, the level of CTFs from the N2a-SwedAPP cells is reduced by treatment with BBR in a concentration-dependent manner, reaching maximal reduction by 55% of basal level at a concentration of 20 μM. At low concentration of BBR (5 μM), there is a 24% reduction in the level of CTFs. Western blot analysis for p-APP in N2a-SwedAPP cell lysates enables the comparison of APP phosphorylation and its processing. Using the anti-p-APP-Thr668 antibody which specifically identifies only this phosphorylated site of the protein to assess BBR's inhibitory effect. There is an apparent decrease in the phopshorylated level of APP by BBR treatment in a similarly dose-dependent fashion as demonstrated by western blot analysis. At 20 μM, the level of p-APP is decreased by 75% and at 10 μM by 50% (FIGS. 11A and 11B). It has been shown by Wang, Y. P., Wang, X. C., Tian, Q., Yang, Y., Zhang, Q., Zhang, J. Y., Zhang, Y. C., Wang, Z. F., Wang, Q., Li, H., Wang, J. Z., 2006. Endogenous overproduction of beta-amyloid induces tau hyperphosphorylation and decreases the solubility of tau in N2a cells. J. Neural Transm. 113, 1723-1732 that in AD models that overexpression of Swedish mutant APP (SwedAPP) increases phosphorylated tau (p-tau). The effect of BBR on abnormal tau phosphorylation at PHF-1 epitope in N2a-SwedAPP cells is then tested. Compared to the control, the level of tau bound to PHF-1 is decreased to 30% and 50% by treatment of 10 and 20 μM concentrations of BBR, respectively (FIGS. 11A and 11B). The data indicates that hyperphosphorylation of tau at Ser-396/404 increases in N2a-SwedAPP cells and the abnormal modification of tau at this epitope was significantly inhibited by BBR.

PI3K has a regulatory role in the trafficking of many proteins including APP (PI3K inhibitors increase intracellular APP-CTFs). Since BBR shows a dose- and time-dependent increase in p-Akt and inactive pGSK3αβ with decrease in p-APP, CTFs and PHF-1 (FIGS. 11A and 11B), whether pretreatment of PI3K inhibitor (LY294002) in N2a-SwedAPP cells abolishe the BBR-induced reduction of p-APP, CTFs and PHF-1 is further determined. To determine the CTFs, p-APP and PHF-1 reducing effect of BBR, N2a-SwedAPP cells are co-treated with 20 μM of BBR together with the PI3K inhibitor, LY294002, used at 20 μM. As expected, BBR-induced reduction of p-APP, CTFs and PHF-1 are reversed by the co-treatment of LY294002 (FIGS. 11C and 11D). To further analyze the BBR effect, p-APP in N2a-SwedAPP cells is further investigated by immunofluorescence. We found that p-APP-positive vesicles could be labeled by the endosome markers Rab5 (FIG. 11E (A1-A4)) as determined earlier (Lee et al. 2003a). After 3h exposure to 20 μM BBR, pAPP staining was markedly reduced in the endosomal compartments (FIG. 11E (B1-B4)). BBR-induced reduction of p-APP was significantly inhibited by LY294002 in endosomal compartments of N2a-SwedAPP cells (FIG. 11E (C1-C4)). This confirms the BBR mediated p-APP reduction seen with Western blots (FIGS. 12c and 12d). These data show that the reduction of p-APP staining inside endosomal compartments is due to BBR's inhibitory effect on p-APP. Altogether, these results indicate that PI3K/Akt/GSK3 is involved in the BBR-induced reduction of p-APP, CTFs and PHF-1. These results also indicate that BBR has an effective role in the regulation of the PI3K/Akt/GSK3 pathway.

Furthermore, recent works such as Beaulieu, Jean-Martin, et. al., Akt/GSK3 Signaling in the Action of Psychotropic Drugs, Annu. Rev. Pharmacol. Toxicol. 2009. 49:327-47, Li X., Tope R. S. 2010. Is glycogen synthase kinase-3 a central modulator in mood regulation? Neuropsychopharmacology 35, 2143-2154, Rockenstein E, Torrance M, Adame A, Mante M, Baron P, et al. (2007) Neuroprotective effects of regulators of the glycogen synthase kinase-3beta signaling pathway in a transgenic model of Alzheimer's disease are associated with reduced amyloid precursor protein phosphorylation. J Neurosci 27: 1981-1991 and Qing H, et al. Valproic acid inhibits Abeta production, neuritic plaque formation, and behavioral deficits in Alzheimer's disease mouse models. J Exp Med. 2008; 205(12):2781-2789 have established the link between the regulation of the PI3K/Akt/GSK3 pathway and means of preventing or treating neural diseases such as neural mood disorders, bipolar disorder, schizophrenia, depression, Tourette syndrome, ADHD, Alzheimer's disease and associated neuropsychiatric conditions.

Discussion

Although BBR is a well-known neuroprotective agent, the actual therapeutic role of BBR in AD pathology has not yet been evaluated. BBR has been shown to inhibit the production of Aβ in H4-SwedAPP cells; however, its effect on Aβ accumulation in vivo has not been documented. The present application demonstrates that regular administration of BBR can prevent the age-related cognitive impairments and Aβ accumulation observed in TgCRND8 mice with an early-onset AD-like pathology. Several lines of our evidence suggest that BBR has beneficial effects in AD. First, BBR readily passes through the blood-brain barrier, and its presence in brain tissue of treated mice is definitively determined by liquid chromatography-mass spectrometry. Second, dose-specific effects on brain Aβ levels, cognitive deficits, amyloid neuropathology and accelerated gloisis are found in the TgCRND8 mouse model of Alzheimer's disease. Third, BBR significantly reduces the Aβ and CTFs, probably by down-regulating the phosphorylation of APP and of CTFs via the activation of PI3K/Akt/GSK3 pathway.

Both low and high doses of BBR significantly reduce the cognitive impairment characteristic of AD, both in the conventional reference memory MWM task and memory retention task (probe trial) of the mice (FIG. 2C). These results are consistent with the amelioration of hippocampal-dependent memory function. The cognitive benefits of long-term BBR administration does not involve significant side effects on sensorimotor function, as evidenced by visible platform training (FIG. 2B)

Notably, BBR-induced decreases in memory deficits are accompanied not only by a significant reduction in the amyloid burden but also by an evident plaque fragmentation in the brains of transgenic mice. In particular, Aβ plaques of all three size subsets (<25, 25-50, and 50 μm) are significantly decreased in the brains of BBR-treated TgCRND8 mice (FIG. 3C). The magnitude of this decrease in the number of plaques is similar to the decrease found in brains of mice treated with arundic acid. The 43-63% reduction in amyloid burden observed in this study is also comparable with that found in studies of mice treated with curcumin or ECGC by Lim, G. P., Chu, T., Yang, F., Beech, W., Frautschy, S. A., Cole, G. M., 2001. The curry spice curcumin reduces oxidative damage and amyloid pathology in an Alzheimer transgenic mouse. J. Neurosci. 21, 8370-8377 and Rezai-Zadeh, K., Shytle, D., Sun, N., Mori, T., Hou, H., Jeanniton, D., Ehrhart, J., Townsend, K, Zeng, J., Morgan, D., Hardy, J., Town, T., Tan, J., 2005. Green tea epigallocatechin-3-gallate (EGCG) modulates amyloid precursor protein cleavage and reduces cerebral amyloidosis in Alzheimer transgenic mice. J. Neurosci. 25, 8807-8814. The present application also includes the important finding that BBR administration can reduce vascular amyloids as well as parenchymal amyloids (FIG. 4A). Apart from neuronal Aβ deposition, previous studies reported in Chishti, M. A., Yang, D. S., Janus, C., Phinney, A. L., Home, P., Pearson, J., Strome, R., Zuker, N., Loukides, J., French, J., Turner, S., Lozza, G., Grilli, M., Kunicki, S., Morissette, C., Paquette, J., Gervais, F., Bergeron, C., Fraser, P. E., Carlson, G. A., George-Hyslop, P. S., Westaway, D., 2001. Early-onset amyloid deposition and cognitive deficits in transgenic mice expressing a double mutant form of amyloid precursor protein 695. J. Biol. Chem. 276, 21562-21570 and Hawkes C. A., McLaurin, J. A., 2009. Selective targeting of perivascular macrophages for clearance of beta-amyloid in cerebral amyloid angiopathy. Proc. Natl. Acad. Sci. USA 106, 1261-1266 have shown that the pattern of vascular amyloid deposition in leptomeningeal and small cortical blood vessels observed in TgCRND8 mice mirrors that typically found in human age-related cerebral amyloid angiopathy (CAA). BBR treatment had significant effects in reducing ThioS-positive vascular amyloids (FIGS. 4B and 4C), and might reduce CAA through reduction of Aβ production. Effects reported in the present study are similar to the reported effects of nicotine administration in Hellstrom-Lindahl, E., Court, J., Keverne, J., Svedberg, M., Lee, M., Marutle, A., Thomas, A., Perry, E., Bednar, I., Nordberg, A., (2004). Nicotine reduces Abeta in the brain and cerebral vessels of APPsw mice. Eur. J. Neurosci. 19, 2703-2710 where percentage of vessels with Aβ was significantly reduced. Because regular administration of BBR reduces ThioS-positive plaques and insoluble peptides, its effect may be on amyloid fibril formation. There is a mild inhibition of Aβ1-42 fibril formation with an IC₅₀ value of 23.5 μM in vitro. It has also been recently demonstrated in Shi, A., Huang, L., Lu, C., He, F., Li, X., 2011. Synthesis, biological evaluation and molecular modeling of novel triazole-containing berberine derivatives as acetylcholinesterase and β-amyloid aggregation inhibitors. Bioorg. Med. Chem. 19, 2298-2305 that BBR at a concentration of 20 μM showed 36.3% inhibition of Aβ1-42 fibril formation in vitro. However, the effective concentration of BBR used in the in vitro studies, approximately 20 μM, are 23 times less than those found in the brain. Therefore, the exact molecular mechanism of BBR-mediated reduction of Aβ accumulation needs further study.

The strong association of reactive astrocytes and activated microglia with amyloid deposition contributes to the progressive course of AD because of amplification of a large range of proinflammatory molecules that mediate, in part, the neuronal loss detected in AD. It is found that treating TgCRND8 mice with BBR resulted in a 45% reduction in microgliosis and a 54% decrease in astrocytosis (FIGS. 6A and 6B).

A similar marked decrease in activated microglia (57%) is also observed in ibuprofen-treated animals. Our results showing that BBR significantly reduces amyloid deposits along with amyloid plaque-associated reactive microgliosis and astrocytosis are in full agreement with the above view. However, this does not essentially entail a unidirectional relationship between the two events. The observed reduction in gliosis by BBR is more likely to be linked to its inhibition of Aβ. However, BBR appears to suppress neuroinflammatory responses independent of Aβ. Therefore, the putative action of BBR needs to be determined in relation to the mechanisms underlying both gliosis and amyloidosis. Nonetheless, because neuroinflammation is a risk factor for neurodegenerative disease, the anti-inflammatory effect of BBR in the TgCRND8 mice supports the evidence for its therapeutic potential for AD.

It has been demonstrated by Kawarabayashi, T., Younkin, L. H., Saido, T. C., Shoji, M., Ashe, K H., Younkin, S. G., 2001. Age-dependent changes in brain, CSF, and plasma amyloid β protein in the Tg2576 transgenic mouse model of Alzheimer's disease. J. Neurosci. 21, 372-381 that successive extraction of brain homogenates using SDS and FA solubilizes the majority of the total amyloid species from brains of Tg2576 mice. Both low and high doses of BBR significantly reduce both soluble and insoluble Aβ peptides (FA fraction) in the brain of TgCRND8 mice. This reduction in insoluble Aβ peptides is comparable with earlier observed reduction after chronic nicotine administration in Swedish APP mice as reported in Nordberg, A., Hellstrom-Lindahl, E., Lee, M., Johnson, M., Mousavi, M., Hall, R., Perry, E., Bednar, I., Court, J., 2002. Chronic nicotine treatment reduces beta-amyloidosis in the brain of a mouse model of Alzheimer's disease (APPsw). J. Neurochem. 81, 655-658. The earlier observation that the nicotine treatment reduced the insoluble but not soluble Aβ peptides in the brain of Tg2576 is in accordance with this result. However, a low dose of BBR reduces SDS-soluble Aβ1-40 (44% reduction); therefore, BBR may also have an effect on potentially neurotoxic soluble Aβ oligomers. Overall, the low dose BBR-induced reduction of total extracted Aβ40 (44-49%) and Aβ42 (22-43%) (FIGS. 5A and 5B) in the brain hemispheres corresponds well with the decrease in plaque number (37%) and plaque load (61%) in other brain hemispheres evaluated by immunostaining, indicating that these biochemical measures are an exact reflection of overall amyloid load (FIGS. 3 and 6). BBR treatment prevents spatial memory reference deficits and Aβ pathology in vivo at doses that are equivalent to or lower than the doses prescribed for humans for hyperchloestermia. The low dosage of BBR shows more significant effect than the high dosage in reducing Aβ pathology and gliosis (FIGS. 3 and 6). A similar case has been published in Dong, H, Yuede, C. M., Coughlan, C., Lewis, B., Csernansky, J. G., 2008. Effects of memantine on neuronal structure and conditioned fear in the Tg2576 mouse model of Alzheimer's disease. Neuropsychopharmacol. 33, 3226-3236, reporting the Aβ-reducing effect of memantine in Tg2567 AD mice. Thus, it is possible that lower BBR doses may obstruct just enough neuronal activity to reduce amyloid generation, while higher dose obstructs so much neuronal activity that Aβ clearance mechanisms are also reduced.

A possible mechanism of the reducing effect of BBR on Aβ deposition is modulation of APP processing, because the levels of APP-CTFs, the direct precursor of Aβ are decreased by BBR treatment (FIG. 4A). APP processing can be modulated either by an altered APP expression or by the function of β-secretase (BACE-1). However, no significant difference in the protein levels of Fl-APP or BACE-1 between the groups is noted (FIG. 7A). It is known that APP phosphorylation at position Thr668 facilitates the accumulation of CTFs and increases Aβ generation. Several studies such as da Cruz e Silva, E. F., da Cruz e Silva O. A. B., 2003. Protein phosphorylation and APP metabolism. Neurochem. Res. 28, 1553-1561 and Lee, M. S., Kao, S. C., Lemere, C. A., Xia, W., Tseng, H. C., Zhou, Y, Neve, R., Ahlijanian, M. K., Tsai, L. H., 2003a. APP processing is regulated by cytoplasmic phosphorylation. J. Cell Biol. 163, 83-95 have revealed that APP maturation and targeting for proteolysis requires APP Thr668 phosphorylation. Phosphorylated APP undergoes fast anterograde axonal transport to the nerve terminals, where β- and γ-secretase mediated-cleavage occurs, resulting in Aβ release. This indicates that BBR-mediated reduction of APP phosphorylation might subsequently result in a reduction of mature p-APP in the trans-Golgi compartment and a lesser amount of APP directed to the distal axon, which would prevent Aβ production at synaptic terminals. These findings show that BBR modulates APP processing through a mechanism beyond regulating the expression of APP and BACE-1. This is consistent with a previous study by Ding, Y, Qiao, A., Wang, Z., Goodwin, J. S., Lee, E. S., Block, M. L., Allsbrook, M., McDonald, M. P., Fan, G. H., 2008. Retinoic acid attenuates beta-amyloid deposition and rescues memory deficits in an Alzheimer's disease transgenic mouse model. J. Neurosci. 28, 11622-11634 which showed that retionic acid reduced the Aβ pathology mainly via reducing CTFs, p-APP without influencing the levels of Fl-APP and BACE-1. Similarly, Indirubin-3-monoxime, a GSK3β inhibitor isolated from the traditional Chinese medicinal herb Indigo naturalis (Qing Dai in Chinese) reduced Aβ accumulation and tau phosphorylation in APP/PS-lmice without affecting the levels of Fl-APP and BACE-1.

Based on the result that BBR treatment decreased the elevation of APP phosphorylation in TgCRND8 mice, we propose that BBR may prevent APP processing by inhibiting its phosphorylation. Among the several protein kinases phosphorylating APP at Thr668, GSK is considered to be a key kinase responsible for APP phosphorylation in neuronal cells. Suppressing GSK3 activity has been demonstrated to decrease the generation and accumulation of Aβ in APP mice. This coincides with our result showing a concomitant down-regulation of GSK activity by BBR treatment in TgCRND8 mice. In TgCRND8 mice and N2a-SwedAPP cells, BBR treatment led to a trend of increased p-Akt and inactive pGSK3α/β protein levels (FIGS. 7E, 7F and 10). GSK3 activity is inhibited via phosphorylation at specific serine residues (Ser 9 and Ser21) by Akt. It is also possible that the improved spatial learning can be ascribed to the BBR-induced activation of the Akt pathway because activation of the Akt pathway is essential for the expression of long-term potentiation. It is also known that the PI3K/Akt/GSK3β signaling pathway contributes to a number of aspects of AD pathology. In vitro studies by Martin, D., Salinas, M., López-Valdaliso, R., Serrano, E., Recuero, M., Cuadrado, A., 2001. Effect of the Alzheimer amyloid fragment Abeta(25-35) on Akt/PKB kinase and survival of PC12 cells. J. Neurochem. 78, 1000-1008 suggested that PI3K and Akt product neurons against Aβ toxicity. GSK3α also modulates APP processing thereby influencing the production of Aβs. Liu, S. J., Zhang, A. H., Li, H. L., Wang, Q., Deng, H. M., Netzer, W. J., Xu, H., Wang, J. Z., 2003. Overactivation of glycogen synthase kinase-3 by inhibition of phosphoinositol-3 kinase and protein kinase C leads to hyperphosphorylation of tau and impairment of spatial memory. J. Neurochem. 87, 1333-1344 showed that inhibition of PI3K contributes to tau phosphorylation and impairment of spatial memory. GSK3β is strongly involved in tau phosphorylation at various sites including PHF-1, Tau-1, AT8 and AT270 epitopes. It has been shown by Ryder, J., Su, Y., Ni, B., 2004. Akt/GSK3beta serine/threonine kinases: evidence for a signaling pathway mediated by familial Alzheimer's disease mutations. Cell Signal 6, 187-200 that GSK3β activity increases in cell expressing Swedish APP mutation and in AD presenilin-1 and presnilin-2 mutation lymphoblast cells via a down-regulation of both Ser473 Akt and inactive Ser9 phosphorylated GSK3β. This application evaluates the involvement of PI3K/Akt within the complex intracellular signaling pathway involved in the inhibition of APP processing. Pharmacological inhibition of the kinase activity of PI3K not only reverses the BBR-induced reduction of p-APP and PHF-1, but it also blocks the BBR-mediated reduction of CTFs (FIG. 6B). Detection of reduced p-APP staining in the endosomal compartment of N2a-SwedAPP cells in immunohistochemistry (FIG. 11C), illustrates that the phosphorylation state of APP is an important factor due to its association with BACE-1, which mediates the amyloidogenic processing of APP to increase Aβ through increased generation of the CTFs. Interference in PI3K/Akt/GSK3 pathway thus represents a key mechanism through which BBR may decrease AD pathology and improve cognitive function.

In conclusion, BBR-comprising composition and method of using the same of the present invention is able to reduce cerebral Aβ levels, glial activation, and cognitive impairment in the TgCRND8 mouse model. In addition, BBR suppresses both CTFs and p-APPs levels via activating the PI3K/Akt/GSK3 signaling pathway, thereby precluding Aβ generation. Based on the safety and brain bioavailability of BBR, and its ability suppress to Aβ levels, it is a promising drug for the prevention and/or treatment of Alzheimer's disease. The outcomes from our study will warrant further investigation of BBR-like alkaloids as candidates for Aβ and tau-based therapeutics to modify or delay the onset of Aβ and tau pathology in AD.

### INDUSTRIAL APPLICABILITY

The present invention discloses a berberine alkaloid and to their use as therapeutic agent, particularly in the treatment of neural disease. In particular, the invention also discloses a treatment directed to therapeutic effect of berberine on Alzheimer's disease by reducing Aβ deposits, tau hyperphosphorylation, gliosis and cognitive impairments.

If desired, the different functions discussed herein may be performed in a different order and/or concurrently with each other. Furthermore, if desired, one or more of the above-described functions may be optional or may be combined.

While the foregoing invention has been described with respect to various embodiments and examples, it is understood that other embodiments are within the scope of the present invention as expressed in the following claims and their equivalents. Moreover, the above specific examples are to be construed as merely illustrative, and not limitative of the reminder of the disclosure in any way whatsoever. Without further elaboration, it is believed that one skilled in the art can, based on the description herein, utilize the present invention to its fullest extent. All publications recited herein are hereby incorporated by reference in their entirety.

What we claim:

1. A composition for prevention or treatment of neural diseases in a subject in needs thereof comprising a therapeutically effective amount of compound of formula I: 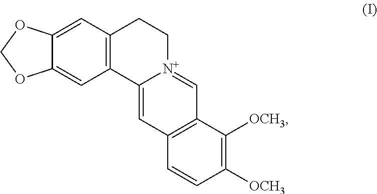 [SMILES: COc1ccc2cc3[n+](cc2c1OC)CCc1cc2c(cc1-3)OCO2]
or generically called Berberine (BBR), the salts and/or derivatives thereof, or a combination thereof, wherein said compound is a kind of isoquinoline alkaloid isolated from a natural source.

2. The composition of claim 1, wherein the neural diseases are diseases associated with abnormal protein aggregation or deposit in the nervous system, wherein said abnormal protein aggregation or deposit is generated by accumulation of amyloid β-peptide (Aβ), tau hyperphosphorylation and gliosis.

3. The composition of claim 1, wherein said composition diminishes said amyloid β-peptide by inhibiting generation of C-terminal fragments of amyloid precursor protein (APP) or phosphorylation of APP.

4. The composition of claim 2, wherein the abnormal protein aggregation or deposit comprising PHF-1, AT8 or AT180 and said abnormal protein aggregation or deposit is found in the brain.

5. The composition of claim 1, wherein the neural diseases comprising neural degenerative diseases, neural mood disorders, bipolar disorder, schizophrenia, depression, Tourette syndrome, ADHD and neuropsychiatric conditions.

6. The composition of claim 5 wherein the neural degenerative diseases comprising Alzheimer's disease, cerebreal amyloid angiopathy and dementia.

7. The composition of claim 1, wherein said subject is a mammal and the therapeutically effective amount of said compound ranges from 25 to 100 mg per Kg of said mammal's body weight per day.

8. The composition of claim 1, wherein said subject is a mammal and the therapeutically effective amount of said compound is 25 mg per Kg of said mammal's body weight per day.

9. The composition of claim 1, wherein said subject is human.

10. The composition of claim 1, wherein said compound of formula I, the salts and derivatives thereof is one or more selected from berberine chloride, berberine hydrochloride, berberine sulfates, berberrubine, palmatine chloride, palmitine, oxyberberine, epiberberine, dihydroberberine, tetrahydroberberine, jatrorrhizine, and/or coreximine.

11. The composition of claim 1, wherein the natural source is a herbal plant.

12. The composition of claim 11, wherein the herbal plant is selected from Coptis chinensis or Berberis species

13. The composition of claim 1, wherein the compound is administered orally.

14. The composition of claim 1 wherein the prevention or treatment of neural diseases is dependent on a PI3K/Akt/GSK3 pathway.

15. A method for prevention or treatment of neural diseases comprising:
administrating to a subject a composition comprising a therapeutically effective amount of compound of formula I: 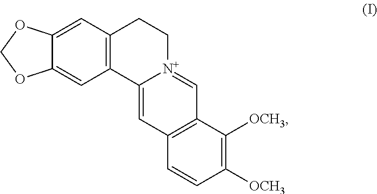 [SMILES: COc1ccc2cc3[n+](cc2c1OC)CCc1cc2c(cc1-3)OCO2]
or generically called berberine (BBR), the salts and derivatives thereof, or a combination thereof;
said compound is a kind of isoquinoline alkaloid isolated from a natural source selected from Coptis chinensis or Berberis species; and the therapeutically effective amount of said compound ranges from 25 to 100 mg per Kg of said subject's body weight per day.
